SEQUENCE LISTING (9 sequences)

SEQ ID NO: 1 - protein, 304 aa, Methylobacterium extorquens
AGETFRLGVLPFGTASWEAAVIKARGFDTANGFTLDIVKLAGNDAARIAFLGGQVDAIVGDLIFAARLGNEGRGVRFSPYSTTEGALMVPAGSPITDLKGLAGKRLGVAGGALDKNWILLRAQARETAGLELENVAQIAYGAPPLLAQKLETGELDAALLYWQFCARLEAKGFKRLISADDVMRAFGAKGAVSLIGYLYEGHTVADRGEVVRGFARASAAAKDALANEPALWETVRPLMAAEDDATFATLKRDFLAGIPRRPIAAERADGERIYAALDRLAGAQLLGVGKSLPPDLYLDASGNG

SEQ ID NO: 2 - DNA, 21 bp, Synthetic construct
  source 1..21: mol_type = other DNA
catcatcaccatcatcactaa

SEQ ID NO: 3 - DNA, 15 bp, Synthetic construct
  source 1..15: mol_type = other DNA
accattgccagacgc

SEQ ID NO: 4 - DNA, 22 bp, Synthetic construct
  source 1..22: mol_type = other DNA
gcccttgacgcgaactggattc

SEQ ID NO: 5 - DNA, 18 bp, Synthetic construct
  source 1..18: mol_type = other DNA
ccctccggcgacgccaag

SEQ ID NO: 6 - DNA, 25 bp, Synthetic construct
  source 1..25: mol_type = other DNA
gcccttgacgacaactggattctgc

SEQ ID NO: 7 - DNA, 18 bp, Synthetic construct
  source 1..18: mol_type = other DNA
ccctccggcgacgccaag

SEQ ID NO: 8 - DNA, 126 bp, Synthetic construct
  source 1..126: mol_type = other DNA
ggtgtaggcaaatccttaccaccggacctgtatttagatgcgtctggcaatggtgagaacctgtacttccagagccatcatcaccatcatcactaagaattcgagctccgtcgacaagcttgcggc

SEQ ID NO: 9 - protein, 331 aa, Methylobacterium extorquens
MLSRRGMLATAALSLAAMLPMARRAQAAGETFRLGVLPFGTASWEAAVIKARGFDTANGFTLDIVKLAGNDAARIAFLGGQVDAIVGDLIFAARLGNEGRGVRFSPYSTTEGALMVPAGSPITDLKGLAGKRLGVAGGALDKNWILLRAQARETAGLELENVAQIAYGAPPLLAQKLETGELDAALLYWQFCARLEAKGFKRLISADDVMRAFGAKGAVSLIGYLYEGHTVADRGEVVRGFARASAAAKDALANEPALWETVRPLMAAEDDATFATLKRDFLAGIPRRPIAAERADGERIYAALDRLAGAQLLGVGKSLPPDLYLDASGNG

(19) United States
(12) Patent Application Publication
(10) Pub. No.: US 2026/0071240 A1
(43) Pub. Date: Mar. 12, 2026

(54) METALLOENZYMES AND CATALYTIC OXIDATION OF METHANOL

(71) Applicant: THE BOARD OF TRUSTEES OF THE UNIVERSITY OF ILLINOIS, Urbana, IL (US)

(72) Inventors: Lisa OLSHANSKY, Champaign, IL (US); Peter J. THOMPSON, Urbana, IL (US)

(73) Assignee: THE BOARD OF TRUSTEES OF THE UNIVERSITY OF ILLINOIS, Urbana, IL (US)

(21) Appl. No.: 19/323,806

(22) Filed: Sep. 9, 2025

Related U.S. Application Data
(60) Provisional application No. 63/692,884, filed on Sep. 10, 2024.
(60) Provisional application No. 63/876,376, filed on Sep. 5, 2025.

(51) Int. Cl. C12P 7/00; B01J 31/00; C02F 1/00; C02F 1/38; C02F 3/34; C02F 101/20; C12N 9/04

(52) U.S. Cl. CPC C12P 7/00; B01J 31/003; C02F 1/001; C02F 1/385; C02F 3/342; C12N 9/0006; C12Y 101/02007; C02F 2101/20

(57) ABSTRACT

Discovery of the first rare earth-dependent enzyme in methylotrophic M. extorquens AM1 prompted research toward understanding the unique chemistry at play in these systems. This enzyme, an alcohol dehydrogenase (ADH), features a La³⁺ ion closely associated with redox-active coenzyme pyrroloquinoline quinone (PQQ). AM1 also produces a periplasmic PQQ-binding protein characterized by a Lys residue hydrogen-bonded to PQQ. Accordingly, we prepared K₁₁₅A-, and K₁₁₅D-PqqT variants to assess the relevance of this site toward metal binding. Isothermal titration calorimetry experiments, and titrations monitored by UV-vis absorption and emission spectroscopies support that K₁₁₅D-PqqT binds tightly (K_{d}=0.6±0.2 μM) to La³⁺ in the presence of bound PQQ and produces spectral signatures consistent with those of ADH enzymes. Addition of benzyl alcohol to La³⁺-bound PQQ⊂K₁₁₅D-PqqT produces spectroscopic changes associated with PQQ reduction, and chemical trapping experiments reveal the production of benzaldehyde, supporting ADH activity.

### RELATED APPLICATIONS

This application claims priority under 35 U.S.C. § 119(e) to U.S. Provisional Patent Application No. 63/692,884, filed Sep. 10, 2024, and U.S. Provisional Patent Application No. 63/876,376, filed Sep. 5, 2025, which applications are incorporated herein by reference.

### REFERENCE TO SEQUENCE LISTING

The present application includes a Sequence Listing in electronic format as an xml file titled “500.162US1_SL” which was created on Sep. 8, 2025 and has a size of 9,729 bytes. The contents of xml file 500.162US1_SL are incorporated herein by reference.

### BACKGROUND OF THE INVENTION

The first rare earth-dependent enzyme was reported in 2011 (J. Biosci. Bioeng. 2011, 84, 547). This and related discoveries that rare earth elements play critical roles in methylotrophic bacteria such as Methylobacterium extorquens AM1, have stimulated interest in the enzymology operative in these organisms. The resulting research has enhanced our understanding of lanthanide (Ln³⁺) homeostasis and the proteins associated with it. Proteins such as lanmodulin, XoxF, XoxJ, XoxG, and PqqT are all upregulated in M. extorquens AM1 in the presence of Ln³⁺ (3). Lanmodulin binds Ln³⁺ with picomolar affinities, but its biological role remains to be established. XoxF has been reported as a methanol dehydrogenase (MDH) that incorporates pyrroloquinoline quinone (PQQ) and La³⁺ in its active site, while XoxJ and XoxG are accessory proteins proposed to activate XoxF and serve as a cytochrome, respectively. The protein PqqT, a periplasmic binding protein with nanomolar affinity for PQQ, is proposed to aid in the transport of exogenous PQQ to the cytosol.

Alcohol dehydrogenase (ADH) enzymes play a central role in the single carbon metabolism of methylotrophic bacteria. Along with the PQQ cofactor, they incorporate either Ca²⁺ (in MxaF), or La³⁺ (in XoxF) within their active sites, depending on whether La³⁺ is present. Given their unusual metal ion dependencies, there is much interest in understanding the structure-function correlations and mechanistic implications of the presence of rare earth elements in MDH enzymes. However, several challenges to these studies exist, including: i) these enzymes typically (though not always, 9) must be expressed within suitable methylotrophic bacteria and provide significantly lowered yields relative to common heterologous protein expressions in E. coli, ii) they depend on accessory proteins for in vivo activation, iii) growth conditions must be carefully controlled for metal content, and iv) their genetic manipulation requires unique codon usage and careful attention to promoter design. Accordingly, artificial metalloproteins (ArMs) have the potential to serve as useful model systems for interrogation of mechanistic and structural contributions to catalysis. Using simplified biological scaffolds equipped with installed or engineered metallocofactor binding sites, ArMs enable a combination of mutagenesis and synthesis, providing a unique set of tunable parameters for systematic study. Recent decades have seen the emergence of many ArM constructs with protein hosts such as azurin, streptavidin, nitrobindin, and heme-containing proteins. De novo designed ArMs have also included α-helical bundles and rare earth-binding TIM barrels.

Currently, the industry standard to convert methanol to formaldehyde, a necessary intermediate in the petrochemical sector, leverages an oxide iron molybdate catalyst. Silver catalysts are being used and they require high temperatures for selectivity which increases the cost of production due to the energy costs. The oxide iron molybdate catalyst has proven to be more efficient but requires high pressure and a temperature range of 300-700 C. Accordingly, there is a need for a catalyst to help mitigate costs of alcohol oxidation by performing the reaction at ambient temperatures and pressures in aqueous solutions.

### SUMMARY

We have developed an extremely robust artificial metalloenzyme (ArM) that mimics methanol dehydrogenase (MDH). However, unlike naturally occurring MDHs, our ArM can be produced on large scale from expression in E. coli. These biocatalysts convert alcohols to aldehydes. The conversion of methanol to formaldehyde occurs on a >20 million ton per year scale annually with an >$30 billion annual market value. At present, this process requires high (300-700 C) temperatures and represents a massive energy sink in the chemical industry. In contrast, our ArM catalyst operates at room temperature and in an oxygen atmosphere. The platform is extremely proficient at catalyzing the conversion of benzyl alcohol to benzaldehyde, and this invention will have important industrial applications after some further optimization to go from benzyl alcohol to catalytic conversion of methanol to formaldehyde under mild (neutral water, pH 7, room temperature, in the presence of atmospheric oxygen) conditions.

Herein, we present a new ArM platform prepared in PqqT, a periplasmic PQQ-binding protein. Through structure-guided mutagenesis, and subsequent biophysical and spectroscopic characterization, we have developed this ArM such that weak and nonspecific metal binding affinities observed in WT-PqqT are converted to biomimetic La³⁺-binding in the developed variant. Obtaining the crystal structure of this protein informed the placement of targeted point mutations (i.e., a non-naturally occurring substitution) for the development of this new ArM scaffold. In the presence of La³⁺, the spectroscopic signatures of the PQQ cofactor undergo distinct changes consistent with previously established PQQ-Mⁿ⁺ interactions (Mⁿ⁺=Ln³⁺, or Ca²⁺) and biochemical studies on ADH. In the presence of benzyl alcohol, this engineered ArM produces benzaldehyde, providing proof-of-concept ADH reactivity. This system represents a rare earth-dependent ArM and features a topical and relevant biomimetic architecture to be exploited in future mechanistic studies and in the development of novel biocatalysts.

Accordingly, this disclosure provides an artificial metalloenzyme comprising:
a) a coordinated metal ion;
b) a pyrroloquinoline quinone (PQQ) redox cofactor;
c) an artificial periplasmic PQQ-binding protein (PqqT) engineered from wild type PqqT, wherein:
   i) K₁₁₅ of the wild type PqqT is substituted with D (K₁₁₅D variant), A (K₁₁₅A variant), or H (K₁₁₅H variant); or
   ii) Y₁₆₁ of the wild type PqqT is substituted with W (Y₁₆₁W variant); or
   iii) both Y₁₆₁ and K₁₁₅ of the wild type PqqT are substituted wherein Y₁₆₁ is substituted with W and K₁₁₅ is substituted with D;

wherein PQQ is hydrogen bonded inside a cleft of the artificial PqqT.

This disclosure also provides a method for catalytic oxidation of an alcohol comprising: contacting an alcohol with an artificial metalloenzyme described above;
wherein the alcohol enters an active site of the artificial metalloenzyme and is thereby catalytically oxidized to an aldehyde.

Additionally, this disclosure provides a method for selectively separating a metal ion from waste comprising contacting a metal ion waste solution with an artificial bacterium, wherein the artificial bacterium comprises:
a) a pyrroloquinoline quinone (PQQ); and
b) an artificial periplasmic PQQ-binding protein (PqqT) engineered from wild type PqqT, wherein K₁₁₅ of the wild type PqqT is substituted with D, and PQQ is hydrogen bonded inside a cleft of the artificial PqqT;

wherein a metal ion from the metal ion waste solution selectively binds to the artificial bacterium and is thereby selectively separated from other metal ions in the metal ion waste solution.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. A lysine-mediated PQQ interaction in PqqT is similar to ion-PQQ interactions in alcohol dehydrogenases. A) The structure of the periplasmic (solute) binding protein PqqT (PDB 9B1U) illuminates the binding interactions that hold PQQ in the canonical ligand binding cleft. B) A strained lysine (top panel) interacts with PQQ in a way that is reminiscent of a lanthanum interaction in the PQQ dependent alcohol dehydrogenases (bottom panel, XoxF PDB 6DAM). C) A topology diagram of PqqT illustrates two domains connected by two polypeptide chains, a characteristic feature that defines group 2 periplasmic binding proteins. The binding pocket of PQQ is circled in grey, and Lys115, which occupies the same position as La3+ or Ca2+ in PQQ dependent dehydrogenases is positioned following β6.

FIG. 2A-H. Titrations monitored by UV-vis absorbance spectroscopy and ITC in which Ca²⁺ (top row) or La³⁺ (bottom row) were added to PQQ bound to either WT-PqqT (left four panels) or K₁₁₅D-PqqT right four panels). Integrated ITC data (●) were fit to an independent binding model ( [symbol] ) and the data depicted are representative examples from experiments performed in triplicate to obtain the thermodynamic parameters reported in Table 1. Insets of UV-vis titrations report relative absorbance intensities at 360 nm (●) for PQQ⊂WT-PqqT (A and E), 320 nm (●) and 360 nm (●) for Ca²⁺ into PQQ⊂K₁₁₅D-PqqT (C), and 352 nm (●) and 383 nm (●) for La³⁺ into PQQ⊂K₁₁₅D-PqqT (G).

FIG. 3. The thermodynamic parameters associated with Ca²⁺ and La³⁺ binding to PQQ⊂PqqT variants were assessed by isothermal titration calorimetry (ITC) at 298 K. Bars represent the average values obtained from triplicate measurements on independently prepared samples, with error bars shown for ±1 s.d.

FIG. 4. UV-vis absorbance changes associated with PQQ reduction upon addition of benzyl alcohol to La³⁺-bound PQQ⊂K₁₁₅D-PqqT. Assay mixtures containing ±100 μM PQQ, ±100 μM metal ion, 110 μM PqqT and 5 mM BzOH in MOPS buffer at pH 7.0 were incubated at room temperature for 30 min, followed by addition of O-((perfluorophenyl)methyl)hydroxylamine in MeOH to induce precipitation of protein and dissolution of product. After centrifugation, soluble reaction mixtures were subjected to LC-MS analysis and compared to a synthetically prepared standard.

FIG. 5. PQQ forms an extensive H-bonding network with both domains of PqqT. Combined, PQQ is observed to interact with 16 nearby atoms: six interactions are made to water molecules, four interactions are formed between PQQ and the larger domain (green), and six interactions are made between PQQ and the smaller domain (grey). Interactions that are formed within hydrogen bonding distance are indicated with yellow dashed lines. A 2mFo-DFc simulated annealing composite omit electron density map is presented in blue and contoured at 1σ.

FIG. 6A-C. Graphs illustrating Ni (A), Co (B), and Cu (C) binding within a K₁₁₅D mutant.

FIG. 7. Graphs illustrating specific copper binding within a K₁₁₅H mutant and a K₁₁₅H plus Y₁₆₁W double mutant.

FIG. 8. Graph of an EPR spectra that shows a radical signal detected within the K₁₁₅D protein scaffold. Copper oxidase proteins leverage radicals to perform oxidative chemistry within enzyme active sites.

FIG. 9. Illustration of nickel catalyzed reactions. The ONO ligand represents PQQ. Palladium can demonstrate similar reactivity. Iridium can catalyze enantioselective olefin hydrogenation reactions.

### DETAILED DESCRIPTION

The 2011 discovery of the first rare earth-dependent enzyme in methylotrophic M. extorquens AM1 prompted intensive research toward understanding the unique chemistry at play in these systems. This enzyme, an alcohol dehydrogenase (ADH), features a La³⁺ ion closely associated with redox-active coenzyme pyrroloquinoline quinone (PQQ), and is structurally homologous to the Ca²⁺-dependent ADH from the same organism. AM1 also produces a periplasmic PQQ-binding protein, PqqT, which we have now structurally characterized to 1.46-Å resolution by X-ray diffraction. This crystal structure reveals a Lys residue hydrogen-bonded to PQQ at the site analogously occupied by a Lewis acidic cation in ADH. Accordingly, we prepared K₁₁₅A-, and K₁₁₅D-PqqT variants to assess the relevance of this site toward metal binding. Isothermal titration calorimetry experiments, and titrations monitored by UV-vis absorption and emission spectroscopies support that K₁₁₅D-PqqT binds tightly (K_{d}=0.6±0.2 μM) to La³⁺ in the presence of bound PQQ, and produces spectral signatures consistent with those of ADH enzymes. These spectral signatures are not observed for WT- or K₁₁₅A-variants, or upon addition of Ca²⁺ to PQQ⊂K₁₁₅D-PqqT. Addition of benzyl alcohol to La³⁺-bound PQQ⊂K₁₁₅D-PqqT (but not Ca²⁺-bound PQQ⊂K₁₁₅D-PqqT, or La³⁺-bound PQQ⊂WT-PqqT) produces spectroscopic changes associated with PQQ reduction, and chemical trapping experiments reveal the production of benzaldehyde, supporting ADH activity. By creating a metal binding site that mimics native ADH proteins, we present a rare earth-dependent artificial metalloenzyme primed for future mechanistic, biocatalytic, and biosensing applications.

Since the discovery that some methylotrophic bacteria use rare earth-dependent alcohol dehydrogenases (ADHs) to meet their metabolic needs, interest in understanding their metal ion homeostasis and ADH activity has surged. However, the unique biochemistry of these organisms and the enzymes that they produce has limited the feasibility of extensive in vitro studies. We present an artificial metalloenzyme (ArM) featuring a biomimetic La³⁺-PQQ active site that overexpresses in E. coli and is stable under a range of conditions. This ArM selectively binds La³⁺ over Ca²⁺ and catalyzes the conversion of benzyl alcohol to benzaldehyde. It thus provides a platform for mechanistic and structural interrogation toward answering long-standing questions regarding the metal ion selectivity, mechanism, and structure-function correlations of PQQ-dependent ADH enzymes.

Additional information and data supporting the invention can be found in the following publication: PNAS 2024 Vol. 121 No. 33 e2405836121 and its Supporting Information, which are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1947; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wuts, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below.

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system.

The term “heteroaryl” refers to a monocyclic, bicyclic, or tricyclic ring system containing one, two, or three aromatic rings and containing at least one nitrogen, oxygen, or sulfur atom in an aromatic ring.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound.

A notation used herein to represent substitution of an amino acid in wild type PqqT with another amino acid to form an artificial PqqT is X_{abc}Z, where X is the wild type amino acid, abc is the position of X in the protein sequence of wild type PqqT, and Z is the substitute amino acid that replaces X at position abc in the protein sequence of wild type PqqT. X_{abc}Z is thus a notation for an artificial PqqT that represents a non-naturally occurring, targeted point mutation in wild type PqqT. The artificial PqqT can comprise one, two, three, four or more variants; these variants correspond to one, two, three, four or more targeted point mutations in wild type PqqT.

In the natural organism, the protein is produced in the cytosol and then exported to the periplasm of the bacteria where it performs its native function. This export process is signaled to the cellular machinery by the presence of a 27-amino acid tail called a leader sequence. However, when we produce the protein in our lab, we purify it directly from the cytosol. To accomplish this outcome, we modify the gene for the protein, removing the 27-amino acid leader sequence. In this way, its production in the cytosol is not followed by export to the periplasm. Since we work with this form of the protein, we use the corresponding numbering herein, which differs from the original by 27 units.

All amino acid substitutions are described in relation to the wild-type sequence of the PqqT protein of Methylobacterium extorquens as set forth below in SEQ ID NO: 1 (shown without the leader sequence—also see Table 9):

(SEQ ID NO: 1)
AGETFRLGVLPFGTASWEAAVIKARGFDTANGFTLDIVKLAGNDAARIA
FLGGQVDAIVGDLIFAARLGNEGRGVRFSPYSTTEGALMVPAGSPITDL
KGLAGKRLGVAGGALDKNWILLRAQARETAGLELENVAQIAYGAPPLLA
QKLETGELDAALLYWQFCARLEAKGFKRLISADDVMRAFGAKGAVSLIG
YLYEGHTVADRGEVVRGFARASAAAKDALANEPALWETVRPLMAAEDDA
TFATLKRDFLAGIPRRPIAAERADGERIYAALDRLAGAQLLGVGKSLPP
DLYLDASGNG.

### Embodiments of the Technology

1. An artificial metalloenzyme comprising:
   a) a pyrroloquinoline quinone (PQQ) redox cofactor;
   b) an artificial periplasmic PQQ-binding protein (PqqT) engineered from wild type PqqT, wherein K₁₁₅ of the wild type PqqT is substituted with D (K₁₁₅D variant), A (K₁₁₅A variant), or H (K₁₁₅H variant), and PQQ is hydrogen bonded inside a cleft of the artificial PqqT; or
   an artificial periplasmic PQQ-binding protein (PqqT) engineered from wild type PqqT, wherein K₁₁₅ of the wild type PqqT is substituted with D (K₁₁₅D variant) or H (K₁₁₅H variant), and PQQ is hydrogen bonded inside a cleft of the artificial PqqT; or
   an artificial periplasmic PQQ-binding protein (PqqT) engineered from wild type PqqT, wherein Y₁₆₁ of the wild type PqqT is substituted with W (Y₁₆₁W variant), and PQQ is hydrogen bonded inside a cleft of the artificial PqqT; or
   an artificial periplasmic PQQ-binding protein (PqqT) engineered from wild type PqqT, wherein both Y₁₆₁ and K₁₁₅ of the wild type PqqT are substituted wherein Y₁₆₁ is substituted with W and K₁₁₅ is substituted with D, and PQQ is hydrogen bonded inside a cleft of the artificial PqqT;
   c) optionally a second substitution, wherein Y₁₆₁ of the artificial PqqT is substituted with W (Y₁₆₁W variant); and
   d) a coordinated metal ion.

In some embodiments, the metal ion is a transition metal ion, an inner transition metal ion, or a rare earth metal ion. In some embodiments, the PQQ-binding protein comprises a larger 294 amino acid domain, a smaller 108 amino acid domain, and a cleft between the larger and smaller domains. In some embodiments, the metalloenzyme comprises the Y₁₆₁W variant. In some other embodiments, the metalloenzyme comprises the Y₁₆₁W variant and the metal ion is Cu¹⁺ or Cu²⁺.

In yet other embodiments, a variant in the artificial PqqT (i.e., a point mutation in wild type PqqT) is selected from the group consisting of D₁₁₄A, D₁₁₄C, D₁₁₄E, D₁₁₄H, D₁₁₄Q, I₁₉₅A, I₁₉₅C, I₁₉₅F, I₁₉₅L, I₁₉₅P, I₁₉₅S, I₁₉₅T, I₁₉₅V, I₁₉₅W, K₁₁₅C, K₁₁₅D, K₁₁₅H, K₁₁₅Q, K₁₁₅S, N₁₁₆A, N₁₁₆C, N₁₁₆D, N₁₁₆E, N₁₁₆H, N₁₁₆Q, R₄₇K, V₅₉A, V₅₉C, V₅₉F, V₅₉L, V₅₉P, V₅₉S, V₅₉T, V₅₉W, and Y₁₆₁W. In another embodiment, the artificial PqqT comprises a second variant wherein the second variant is Y₁₆₁W. In additional embodiments, an artificial PqqT comprises one or more variants shown in Chart 1.

CHART 1
Examples of an engineered artificial pyrroloquinoline quinone binding protein.
Number of variants in an engineered artificial PqqT

| 1 | 2 | 3 | 4 |
|---|---|---|---|
| D₁₁₄A | D₁₁₄A, Y₁₆₁W | | |
| | K₁₁₅C, Y₁₆₁W | | |
| K₁₁₅D | K₁₁₅D, Y₁₆₁W | K₁₁₅D, R₄₇K, Y₁₆₁W | K₁₁₅D, I₁₉₅C, V₅₉C, Y₁₆₁W |
| K₁₁₅H | K₁₁₅H, Y₁₆₁W | K₁₁₅H, R₄₇K, Y₁₆₁W | |
| K₁₁₅Q | K₁₁₅Q, Y₁₆₁W | | |
| K₁₁₅S | K₁₁₅S, Y₁₆₁W | | |
| R₄₇K | | | |
| Y₁₆₁W | | | |

2. The metalloenzyme of embodiment 1, wherein the redox cofactor is 4,5-dioxo-4,5-dihydro-1H-pyrrolo[2,3-f]quinoline-2,7,9-tricarboxylic acid:

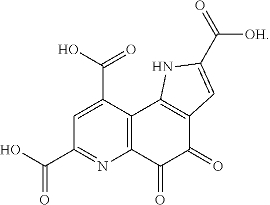 [SMILES: O=C(O)c1cc(C(=O)O)c2c(n1)C(=O)C(=O)c1cc(C(=O)O)[nH]c1-2]

In some embodiments, the redox cofactor has an oxidation state of 1+, 2+, or 3+.

In some embodiments, within the cleft at least six amino acids of the larger domain and the smaller domain hydrogen bond with the carboxylate groups of the redox cofactor at positions C2, C7 and C9.

In some embodiments, three amino acids of the larger domain hydrogen bond with two carboxylate groups of the redox cofactor, wherein A₄₂ and L₂₇ hydrogen bond with a carboxylate group at C2 (A₄₂-C2 and L₃₇-C2), and T₁₄ hydrogen bonds with a carboxylate group at C9 (T₁₄-C9). In some embodiments, three amino acids of the smaller domain hydrogen bond with one carboxylate group of the redox cofactor, wherein G₁₁₂, K₁₁₅, and N₁₁₆ hydrogen bond with a carboxylate group at C7 (G₁₁₂-C7, K₁₁₅-C7, and N₁₁₆-C7). In some embodiments, amino acid K₁₁₅ of the smaller domain hydrogen bonds with a C5-carbonyl and a N6-nitrogen of the redox cofactor.
3. The metalloenzyme of embodiment 1 or 2, wherein the metal ion is a lanthanide or actinide group ion, or a transition metal ion. In various embodiments, the lanthanide group ion has a 3+ formal charge. In some embodiments, the lanthanide group ion has a 4+ formal charge. In various embodiments, the actinide group ion has a 3+ formal charge. In some embodiments, the actinide group ion has a 4+ formal charge.
4. The metalloenzyme of any one of embodiments 1-3, wherein the metal ion is: La³⁺, Gd³⁺, Ce³⁺, Ce⁴⁺, Pr³⁺, Nd³⁺, Pm³⁺, Eu³⁺, Tb³⁺, Ho³⁺, Er³⁺, Tm³⁺, Yb³⁺, Lu³⁺, Y³⁺, Ni²⁺, Co²⁺, Cu²⁺, or Ca²⁺; or
   a metal ion of La, Gd, Ce, Nd, Pr, Pm, Eu, Tb, Dy, Ho, Er, Tm, Yb, Lu, Y, Ca, Fe, Zn, Ti, V, Cr, Mn, Co, Ni, Cu, Mo, Ru, Rh, Pd, W, Re, Os, Ir, Pt, or Au. In some embodiments, the metal ion has a formal charge of 1+, 2+, 3+, 4+, or 5+. In some other embodiments, the metal ion has a formal charge of 2+, 3+, or 4+. In some embodiments, the formal charge of the metal ion is delocalized onto the redox cofactor.
5. The metalloenzyme of any one of embodiments 1-4, wherein the metal ion is La³⁺.

In other embodiments the metal ion is selected from the group consisting of: Ti³⁺, Ti⁴⁺, V²⁺, V³⁺, V⁴⁺, V⁵⁺, Cr²⁺, Cr³⁺, Cr⁴⁺, Cr⁵⁺, Mn¹⁺, Mn²⁺, Mn³⁺, Mn⁴⁺, Mn⁵⁺, Fe¹⁺, Fe²⁺, Fe³⁺, Fe⁴⁺, Fe⁵⁺, Co²⁺, Co³⁺, Ni¹⁺, Ni²⁺, Ni³⁺, Ni⁴⁺, Cu¹⁺, Cu²⁺, Cu³⁺, Zn²⁺, Mo²⁺, Mo³⁺, Mo⁴⁺, Mo⁵⁺, Ru²⁺, Ru³⁺, Ru⁴⁺, Ru⁵⁺, Os²⁺, Os³⁺, Os⁴⁺, Os⁵⁺, Rh¹⁺, Ru²⁺, Ru³⁺, Ru⁴⁺, Ir¹⁺, Ir²⁺, Ir³⁺, Ir⁴⁺, Pd²⁺, Pd⁴⁺, Pt²⁺, and Pt⁴⁺.
6. The metalloenzyme of any one of embodiments 1-5, wherein the metalloenzyme is expressed in a Gram-negative bacterium; or an organism.
7. The metalloenzyme of any one of embodiments 1-6, wherein the metalloenzyme is expressed in E. coli.
8. The metalloenzyme of any one of embodiments 1-7, wherein the pyrroloquinoline quinone and the PQQ-binding protein are bound together in a 1:1 ratio.
9. The metalloenzyme of any one of embodiments 1-8, wherein the metalloenzyme is the K₁₁₅D variant.
10. The metalloenzyme of any one of embodiments 1-9, wherein the metalloenzyme comprises a biomimetic La³⁺-PQQ active site within the artificial periplasmic PQQ-binding protein.
11. A method for catalytic oxidation of an alcohol comprising contacting an alcohol with an artificial metalloenzyme according to any one of embodiments 1-10; wherein the alcohol enters an active site of the artificial metalloenzyme and is thereby catalytically oxidized to an aldehyde.
12. The method of embodiment 11, wherein the metalloenzyme is the K₁₁₅D variant and comprises a biomimetic La³⁺-PQQ active site within the artificial periplasmic PQQ-binding protein.
13. The method of embodiment 11 or 12, wherein the alcohol is methanol or benzyl alcohol.
14. The method of any one of embodiments 11-13, wherein the alcohol is methanol and the methanol is oxidized to formaldehyde.
15. A method for selectively separating a metal ion from waste comprising contacting a metal ion waste solution with an artificial bacterium, wherein the artificial bacterium comprises:
   a) a pyrroloquinoline quinone (PQQ); and
   b) an artificial periplasmic PQQ-binding protein (PqqT) engineered from wild type PqqT, wherein K₁₁₅ of the wild type PqqT is substituted with D, and the redox cofactor (PQQ) is hydrogen bonded inside a cleft of the artificial PqqT;

wherein a metal ion from the metal ion waste solution selectively binds to the artificial bacterium and is thereby selectively separated from other metal ions in the metal ion waste solution.

In some embodiments, the metal ion is a rare earth metal ion. In some embodiments, the selective separation is performed electrochemically. In some other embodiments, the method comprises features of any one of embodiments 1-10.
16. The method of embodiment 15, wherein the metal ion is La³⁺ and the metal ion is selectively separated from other metal ions in the metal ion waste solution. In some embodiments, the metal ion is reduced electrochemically or chemically to a metal.
17. The method of embodiment 15 or 16, further comprising filtering or centrifuging the selectively bound metal ion from the metal ion waste solution.
18. An engineered bacterial cell expressing an exogenous metalloenzyme, the metalloenzyme comprising:
   a) a pyrroloquinoline quinone (PQQ) redox cofactor;
   b) an artificial periplasmic PQQ-binding protein (PqqT) engineered from wild type PqqT, wherein K₁₁₅ of the wild type PqqT is substituted with D or A, and the redox cofactor (PQQ) is hydrogen bonded inside a cleft of the artificial PqqT; and
   c) optionally a metal ion.

In some embodiments the metal ion is a transition metal ion or an inner transition metal ion. In some other embodiments, the engineered bacterial cell comprises features of any one of embodiments 1-10.
19. The bacterial cell of embodiment 18, wherein the bacterial cell is Gram negative.
20. The Gram negative cell of embodiment 19, wherein the bacteria cell is E. coli.
21. An artificial metalloenzyme comprising:
   a) a pyrroloquinoline quinone (PQQ) redox cofactor;
   b) an artificial periplasmic PQQ-binding protein (PqqT) engineered from wild type PqqT, wherein one, two, or more amino acids of the wild type PqqT is substituted with one or more other amino acids, and the redox cofactor (PQQ) is hydrogen bonded inside a cleft of the artificial PqqT; and
   c) a metal ion.

In some embodiments, the artificial metalloenzyme comprises features of any one of embodiments 1-10.
22. A method for catalytic carbon-carbon bond formation comprising:
   contacting an electrophilic alkane, organometallic, or alkene and an organic halide or organic triflate with an artificial metalloenzyme according to any one of embodiments 1-10 or 21;
      wherein the electrophilic alkane, organometallic, or alkene and the organic halide or organic triflate enter an active site of the artificial metalloenzyme and are thereby catalytically coupled together to form a product comprising a new carbon-carbon bond.

In some embodiments the organic halide is an aryl halide. In some embodiments the organic triflate is an aryl triflate. In some embodiments, the metal ions is Ni²⁺, Pd²⁺, Co²⁺, Cu²⁺, or Ir²⁺.
23. The method of embodiment 22, wherein the carbon-carbon bond is formed enantioselectively and the product comprises a new chiral center.
24. A method for catalytic enantioselective olefin hydrogenation comprising contacting an olefin and an artificial metalloenzyme according to any one of embodiments 1-10 or 21 in an atmosphere of hydrogen gas, wherein the olefin and hydrogen gas enter an active site of the artificial metalloenzyme and the olefin is thereby enantioselectively hydrogenated. In some embodiments, the metal ion is Ir²⁺.

### Results

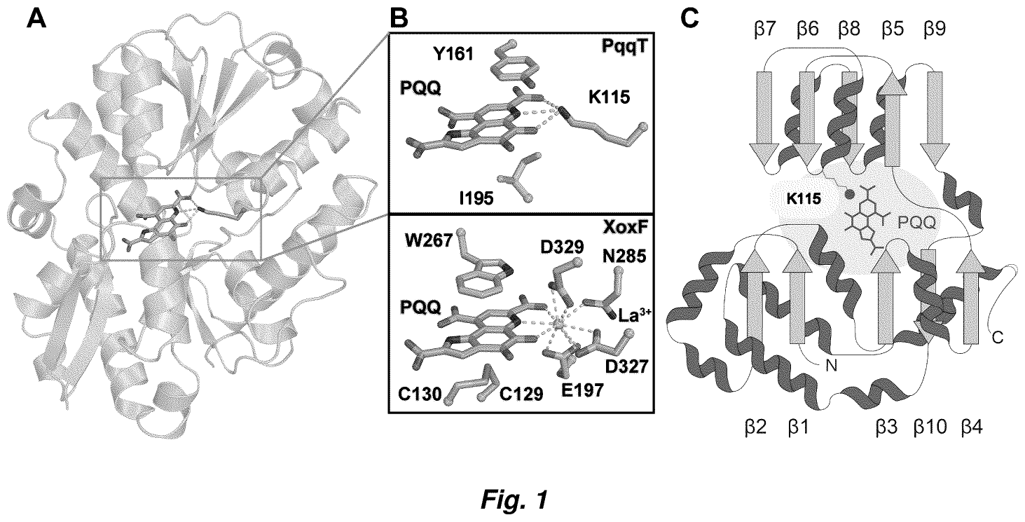

Wild Type Structural Studies by X-ray Diffraction. Optimization of the reported expression protocols for PqqT doubled the yields of purified protein to 120-150 mg per L growth media and enabled crystallization screening. Ultimately, these efforts provided X-ray quality single crystals that provided a 1.46-Å resolution structure of PQQ bound to PqqT (PQQ⊂PqqT). As illustrated in FIG. 1, the solved structure contains a single monomer of PqqT in the asymmetric unit. Based upon the small angle X-ray scattering (SAXS) experiments described below, this monomer likely corresponds to the biological unit of PqqT. This structure reveals that the protein fold of PqqT is starkly different from many other structurally characterized PQQ-bound proteins, which have a propeller fold composed of radially arranged β-sheets. Instead, each PqqT monomer contains two distinct globular domains: a large 294 residue-containing domain and a small 108 residue-containing domain. The large domain is composed of a central five-stranded β-sheet that is adorned on both sides with seven α-helices, and the small domain is comprised of a similar αβα architecture (FIG. 1). However, in this case, the central β-sheet is adorned by just four α-helices.

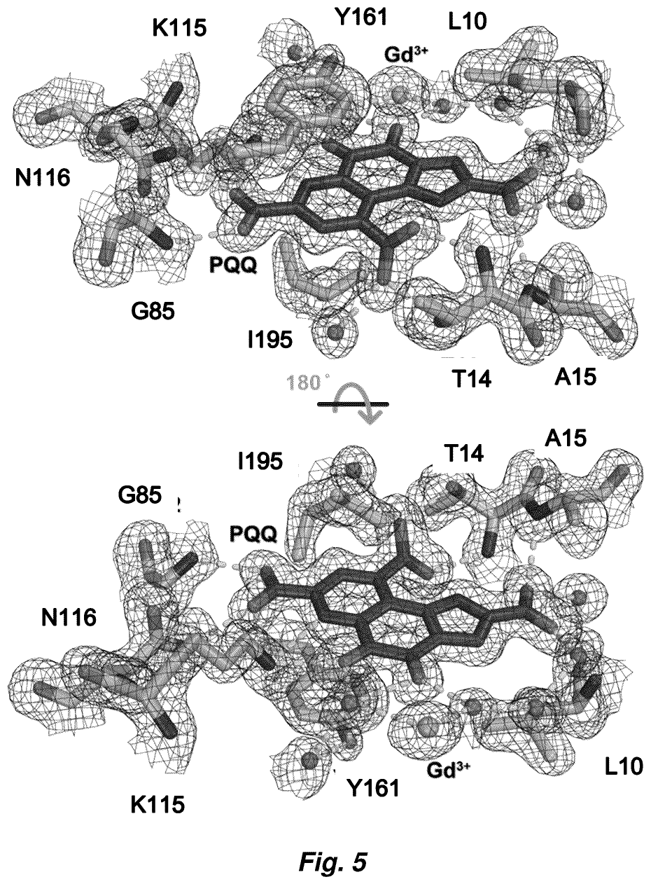

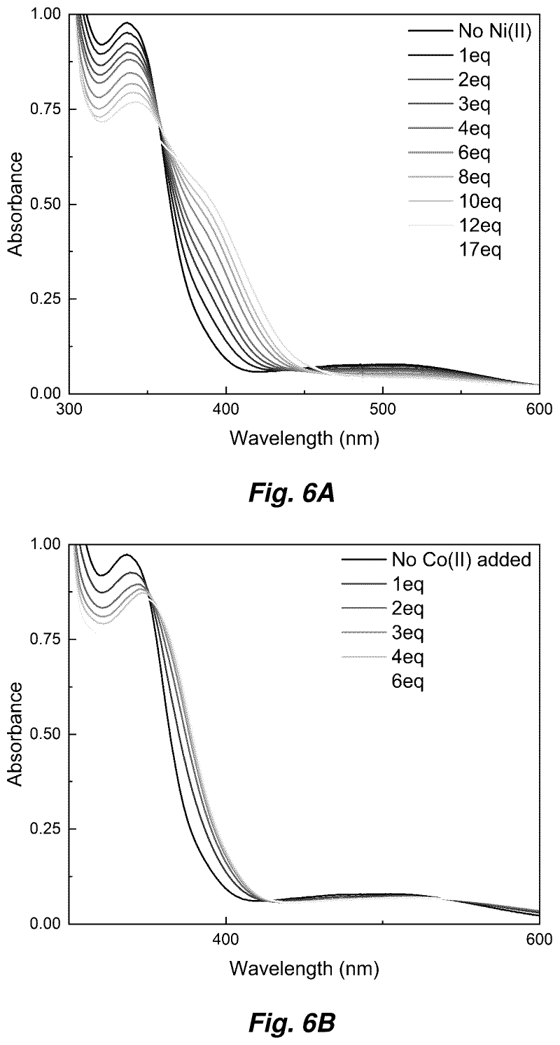

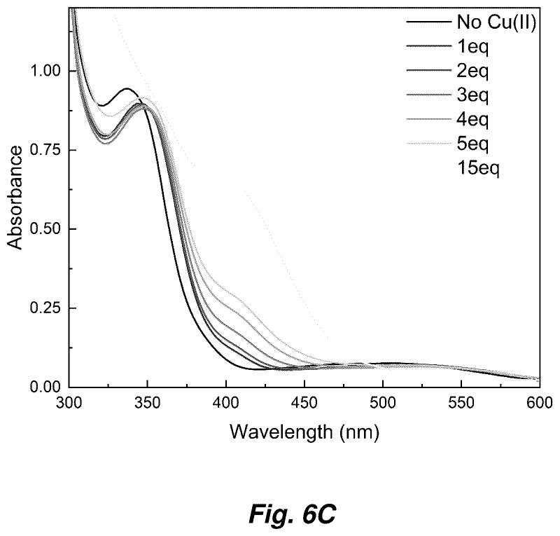

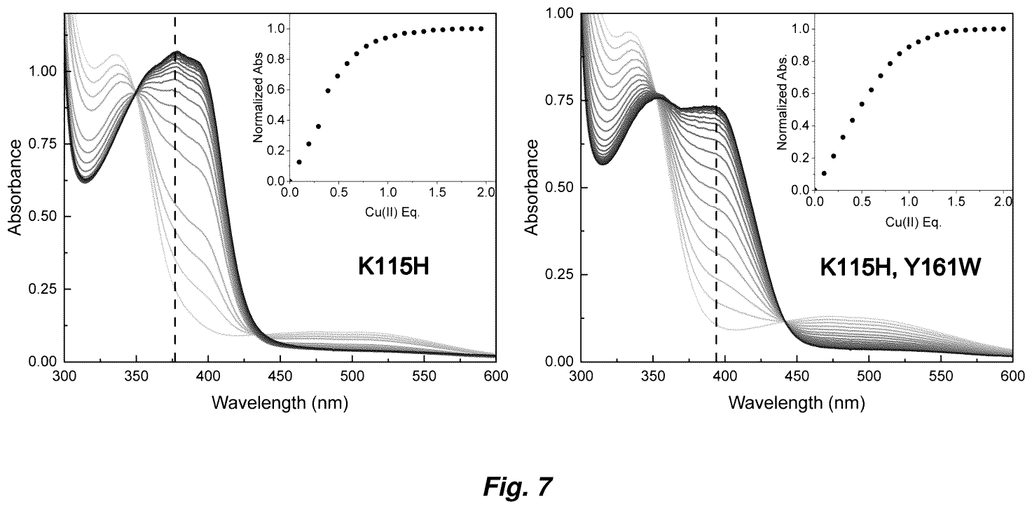

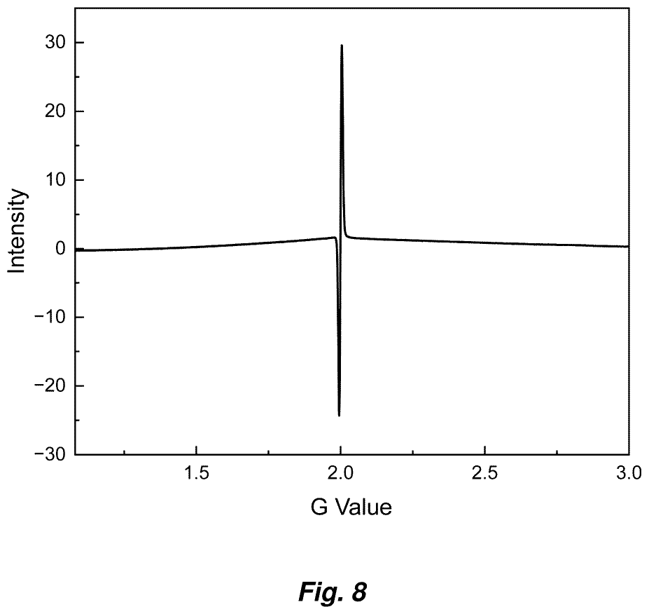

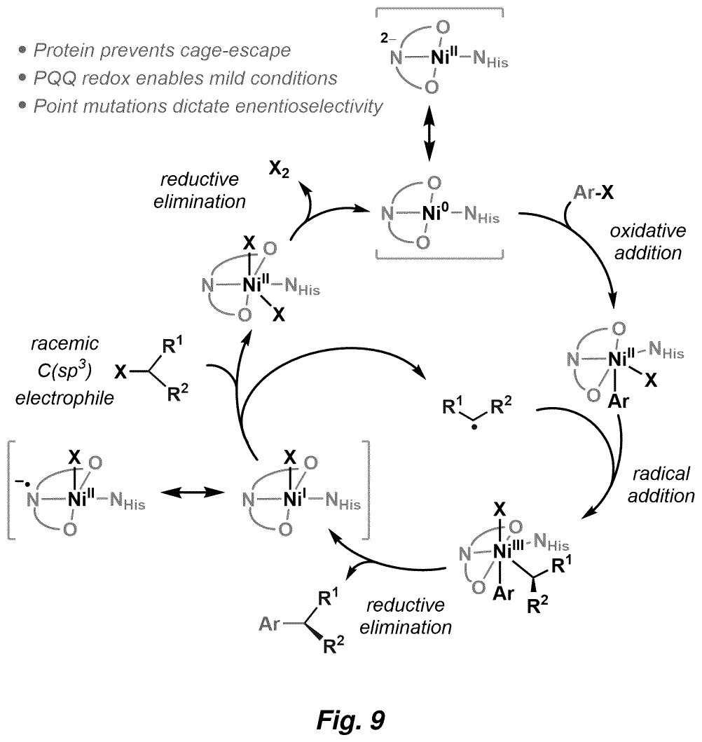

As observed in other categorized “group II” periplasmic binding proteins, the two domains of PqqT are linked by two polypeptide segments and a ligand binding cleft that, in our structure, contains PQQ (FIG. 1). Additionally, PQQ is coordinated by five molecules of water, and residues from both domains. The backbone and sidechain of T₁₄, and the backbone amides of L₁₀ and A₁₅ from the large domain hydrogen-bond (H-bond) with the C9 and C2 carboxylate PQQ functional groups, respectively (FIG. 5). Similarly, the backbone amide of G₈₅ as well as the sidechains of K₁₁₅ and N₁₁₆ from the small domain H-bond with the C7 carboxylate of PQQ.

K₁₁₅ is additionally poised within H-bonding distance of the carbonyl of C5, the carboxylate of O7, and the N6 atoms of PQQ and thus plays a key role in tethering PQQ in this binding site. Analysis of the structure, highlights that to form these three contacts with PQQ, the sidechain of K₁₁₅ adopts a sterically strained, and presumably high-energy, rotameric conformation. Quite interestingly however, this orientation places the positively charged amino group in a position that is reminiscent of where Ca²⁺ in MxaF, and La³⁺ in XoxF coordinate to the carbonyl of C5, the carboxylate of O7, and the N6 atoms of PQQ. Accordingly, it appears to serve as a Brønsted acidic substitute for the Lewis acidic metal ions found in ADH enzymes.

Comparison of these structures also reveals that the π-π interaction between Y₁₆₁ and PQQ is analogous to the conserved interaction in XoxF (W₂₆₇), DepA (W₂₄₁), and MxaF (W₂₇₁). There are also important notable differences within the PqqT binding pocket as compared to these MDHs. For example, rather than I₁₉₅, both MxaF and XoxF position PQQ near a disulfide bridge that is proposed to mediate electron transfer during catalysis. Furthermore, one of the conserved ligands to Ca²⁺ in MxaF, and La³⁺ in XoxF and DepA is a Glu or Gln residue. In PqqT, the backbone of E₈₄ spatially overlays with these residues. However, unlike that observed in MxaF, XoxF, and DepA, the E₈₄ sidechain, which is also a rotameric outlier in the structure, points away from PQQ, a conformation that appears to be imposed by the presence of I₁₉₅.

As described above, the structural solution presented here confirms that PqqT belongs to the periplasmic binding protein (PBP) family. This category of proteins is known for their conformational changes upon ligand binding. To examine ligand-induced structural changes in PqqT, an investigation into the melting temperatures (Tₘ) of PqqT in the presence of PQQ and either Ca²⁺ or La³⁺ was undertaken. Here, it was determined that the Tₘ of PqqT in buffer is approximately 55° C. The addition of PQQ to the protein results in a remarkable (ΔTₘ of 10° C.) increase in protein thermal stability, which is not altered by titration of either Ca²⁺ or La³⁺ into the solution (Table 1). This change upon PQQ binding is consistent with an open to a closed state of PqqT, or as observed in the structure, is simply reflective of the creation of an extended H-bonding network between the two lobes of PqqT and PQQ (FIG. 5).

To complement the thermal stability measurements, SAXS experiments were performed to assess whether conformational changes take place upon PQQ binding. The Guinier fits for these experiments show that PqqT goes from R_{g}=21.14±0.05 Å without PQQ bound to R_{g}=20.53±0.04 Å with PQQ bound. These radii of gyration correlate well with the radius of gyration (19.4 Å) estimated from the crystal structure using HydroPro. The Kratky plots show that in both the bound and unbound states the protein remains in a compact globular state. Finally, a bead model reconstruction of PqqT in solution, aligns well with the solved monomeric unit of the crystal structure, consistent with this protein behaving as a monomeric species in solution.

These results indicate that the two domains form relatively stable interactions even in the absence of PQQ, similar to other periplasmic binding proteins that can populate both open and closed conformations in the absence of the target ligand. The extra intra-domain H-bond interactions formed in response to PQQ binding are likely to restrict conformational flexibility, accounting for the observed changes in thermal stability.

TABLE 1
Thermal stability of PqqT increases with PQQ bound.

| Conditions | Tₘ (° C.)^{a} | ΔTₘ (° C.) |
|---|---|---|
| PqqT | 54.8 ± 0.3 | |
| PqqT + Ca²⁺ | 55.0 ± 0.1 | +0.2 |
| PqqT + La³⁺ | 55.2 ± 0.1 | +0.4 |
| PqqT + PQQ | 64.8 ± 0.1 | +10 |
| PqqT + PQQ + Ca²⁺ | 65.1 ± 0.2 | +10.3 |
| PqqT + PQQ + La³⁺ | 63.9 ± 0.5 | +9.1 |

^{a}Tₘ presented as the average and standard deviation of three technical replicates.

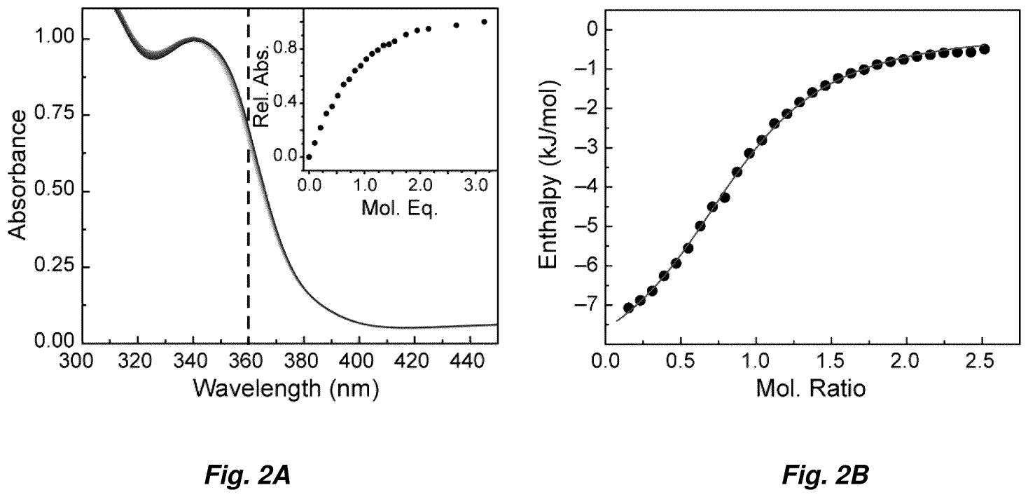

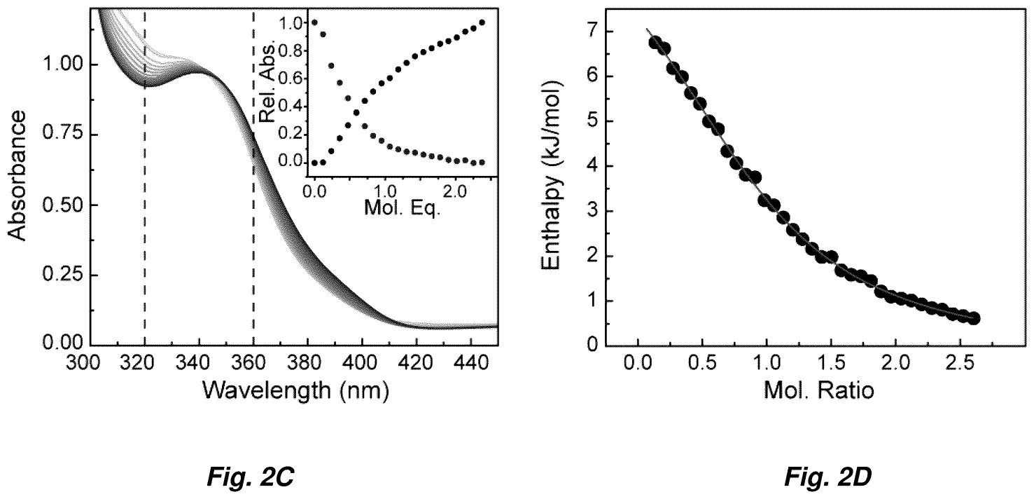

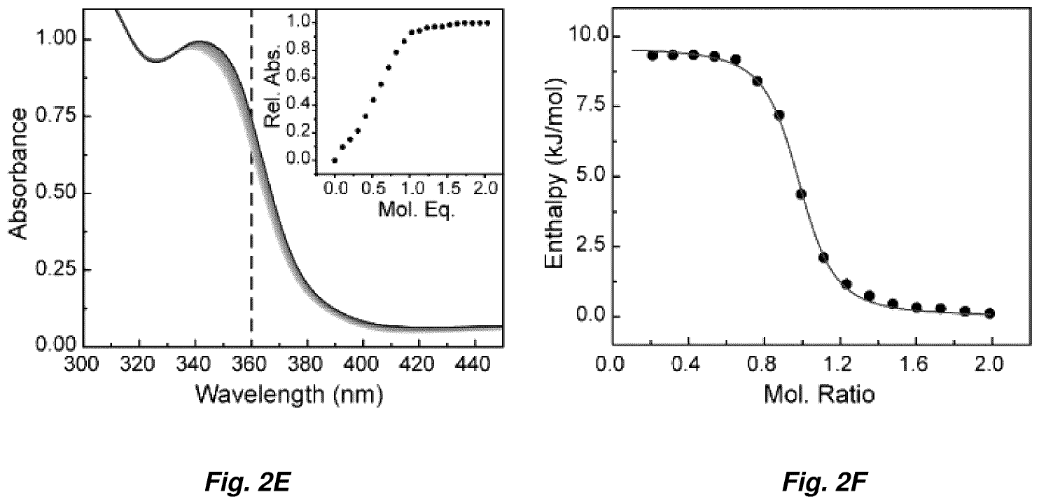

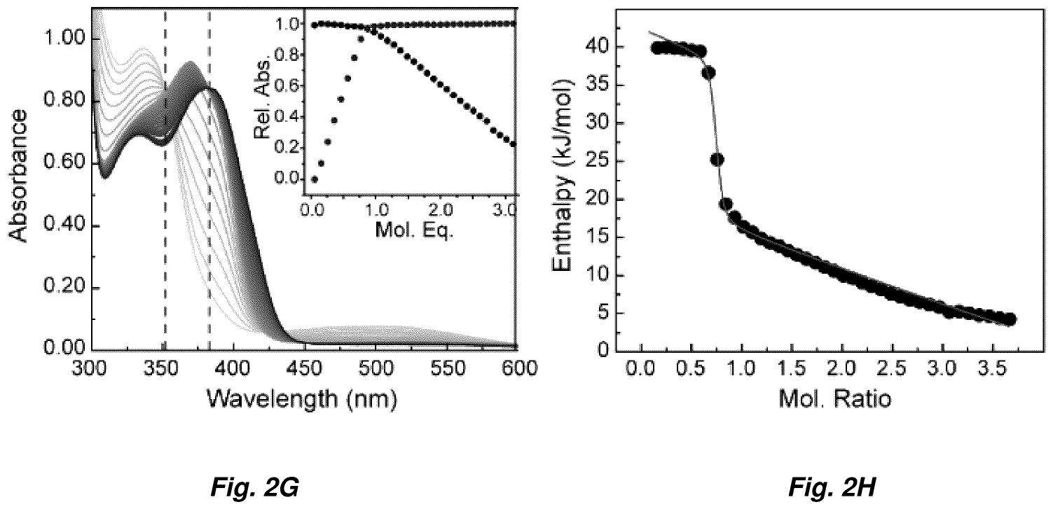

P [symbol] [symbol] ⊂WT-PqqT Binds Weakly to Ln³⁺ and Ca²⁺. Owing to the fact that PqqT transcription in M. extorquens is upregulated in the presence of La³⁺, potential binding interactions between PQQ⊂PqqT and metal ions were investigated by UV-vis absorption spectroscopy and isothermal titration calorimetry (ITC). Specifically, the affinity of PQQ⊂PqqT for Ca²⁺ and La³⁺, the two metal ions operative PQQ-dependent MDHs, were investigated. These studies showed that PQQ⊂WT-PqqT binds both Ca²⁺ and La³⁺ with 1:1 stoichiometries (FIG. 2). ITC data were fit to an independent binding model that incorporates a linear blank function to account for background heats and nonspecific metal binding events. These fits provided K_{d}s of 64±5 μM, and 6±1 μM for Ca²⁺ and La³⁺, respectively (Table 2 and FIG. 2). Addition of La³⁺ to PQQ⊂WT-PqqT revealed an endothermic binding event, while those with Ca²⁺ were exothermic (FIG. 2, Table 2).

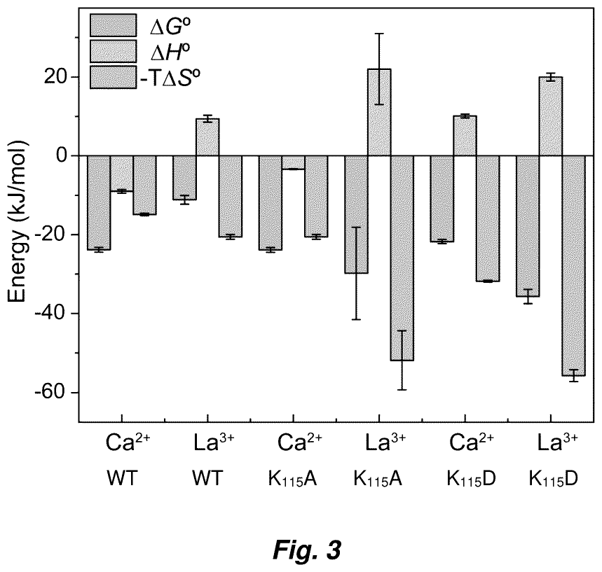

As shown in FIG. 3, analysis of the thermodynamic parameters of metal binding revealed that for La³⁺, a large positive entropy change overcomes a positive enthalpy term such that the thermodynamically favorable binding event is entropically driven. When comparing these parameters for Ca²⁺, the binding event is favorable through a combination of a negative enthalpy and a positive entropy change. In addition to water displacement within the protein fold, the different values observed for ΔS^{o} between the two metal ions may arise from their differences in solvation; Ca²⁺ typically coordinates 6-8 Lewis basic sites in aqueous solution, and exhibits a hydration enthalpy of −1600 kJ/mol, while La³⁺ exhibits coordination numbers of 10-12 and hydration enthalpy of −3300 kJ/mol. Control experiments performed in the absence of PQQ revealed that WT-PqqT binds La³⁺ with a K_{d}=500±350 μM (Table 3), while no interactions were observed with Ca²⁺.

TABLE 2
Metal Ions Binding to PQQ ⊂ WT-, K₁₁₅A-
, and K₁₁₅D-PqqT, as Determined by ITC.^{a}

| Metal | Variant | K_{d} (μM) | ΔH° (kJ · mol⁻¹) | ΔS° (J · mol⁻¹ · K⁻¹) | n |
|---|---|---|---|---|---|
| Ca²⁺ | WT-PqqT | 64 ± 5 | −9.0 ± 0.5 | 50 ± 1 | 0.80 ± 0.05 |
| | K₁₁₅A-PqqT | 60 ± 10 | −3.4 ± 0.1 | 69 ± 2 | 0.77 ± 0.08 |
| | K₁₁₅D-PqqT | 150 ± 30 | 10.1 ± 0.5 | 107.0 ± 0.8 | 0.89 ± 0.07 |
| La³⁺ | WT-PqqT | 6 ± 1 | 9.4 ± 0.9 | 132 ± 3 | 0.97 ± 0.08 |
| | K₁₁₅A-PqqT | 7 ± 5 | 22 ± 9 | 147 ± 25 | 1.04 ± 0.04 |
| | K₁₁₅D-PqqT | 0.6 ± 0.2 | 20 ± 1 | 187 ± 5 | 0.9 ± 0.2 |

^{a}Obtained from fitting protocols described in the SI. Reported uncertainties account for standard error from the fits, as well as ± 1 s.d. for three independently prepared samples.

As shown in FIG. 2, the metal binding titrations examined by ITC were corroborated by titrations monitored by UV-vis absorption spectroscopy. Examining the spectroscopic features associated with PQQ⊂PqqT as a function of added metal ion, subtle yet distinct changes were observed between 320-400 nm. These data supported stoichiometric binding, as illustrated by insets in FIG. 2 reporting absorbance intensity changes at 360 nm.

Intensity changes observed upon metal ion binding are minimal (ΔO.D.=0.1 for addition of 1 eq. Mⁿ⁺) and required these experiments to be performed at high (100 μM) protein concentrations which are inconsistent with the dynamic range of metal binding (Table 2), precluding our ability to obtain K_{d}s directly from these data. As previously reported, PQQ binds these metal ions in the absence of the protein, but the spectral changes observed in the presence of protein are distinct. Moreover, the protein does not absorb across the 320-400 nm range, allowing us to directly probe changes impacting bound PQQ⊂WT-PqqT.

TABLE 3
Binding Thermodynamics Determined by ITC for Addition
of La³⁺ into X-PqqT in the Absence of PQQ.^{a}

| Variant | K_{d} (μM) | ΔH° (kJ/mol) | AS° (J · mol⁻¹ · K⁻¹) | n |
|---|---|---|---|---|
| WT-PqqT | 500 ± 350 | 30 ± 10 | 170 ± 50 | 0.2 ± 0.2 |
| K₁₁₅A-PqqT | 200 ± 100 | 12 ± 6 | 140 ± 10 | 1.1 ± 0.2 |
| K₁₁₅D-PqqT | 25 ± 9 | 23 ± 2 | 166 ± 3 | 1.1 ± 0.1 |

^{a}Values reported were determined as described in the ITC Analysis section above.

Given that XoxF has been shown to function with Ln³⁺ ranging from La³⁺ to Eu³⁺ (with the exception of Pm³⁺), a range of potential metal-binding capabilities were explored by UV-vis absorption spectroscopy. Several other Ln³⁺ ions exhibited 1:1 binding with PQQ⊂WT-PqqT. Tabulating the cumulative results of these studies revealed that WT-PqqT is somewhat indiscriminate toward metal ion selectivity, binding Ca²⁺, La³⁺, Y³⁺, Ce³⁺, Nd³⁺, and Gd³⁺. In addition to the lack of selectivity, the binding affinities determined for Ca²⁺ and La³⁺ are weak relative to most metal-binding proteins, and thus may not be biologically relevant. In support of this suggestion, a Gd³⁺-soaked crystal structure of PQQ⊂WT-PqqT was solved to 1.55-Å resolution. This structure is similar to that of the metal-free structure described above, with a root-mean-square deviation (r.m.s.d.) of 0.09 Å (overlay of 260 Cα atoms). However, unlike the former structure, this structure contains three low-occupancy Gd³⁺ ions. One of these ions is located near the C4-oxygen atom of PQQ, and is ligated by two water molecules, N₇₀, R₄₇, and the backbone carbonyl oxygen of V₅₉. Despite its close proximity to PQQ, this site does not mimic the active sites of XoxF or MxaF. However, its presence close to PQQ provides a potential explanation for the subtle spectral changes observed by UV-vis absorption spectroscopy.

The presence of this ion also highlights an entrance tunnel that connects bulk solvent to K₁₁₅ and thereby highlights a route through which Gd³⁺ may be able to access this site. The second Gd³⁺ ion is found in a distal site near the end of β10, and the third is present between two symmetry related molecules of PqqT. As these ions occupy similar positions to an equivalent set of Na⁺ ions that were modeled into the metal-free structure, they are likely non-specific and present due to the crystal soaking conditions.

Generation of a Biomimetic La³⁺-P [symbol] [symbol] Binding Site. The crystal structure described above reveals that Brønsted acidic K₁₁₅ mimics the interactions with PQQ from Lewis acidic La³⁺ in XoxF and Ca²⁺ in MxaF and DepA. Informed by previously reported mutational studies on MDH enzymes, K₁₁₅A and K₁₁₅D variants of PqqT were prepared. Each of these variants overexpressed well to provide 100-120 mg of purified, de-tagged protein per L of growth media. In good agreement with previous reports (Biochemistry 2019, 58, 2665), the K_{d} for WT-PqqT binding PQQ measured by ITC was 60±40 nM (Table 4). The stoichiometry of PQQ binding was corroborated by titrations monitored by UV-vis absorption and emission spectroscopies, confirming a 1:1 ratio of PQQ:PqqT. Repeating these experiments with the K₁₁₅A and K₁₁₅D variants also revealed 1:1 ratios of PQQ:PqqT, but now with ˜10² larger K_{d}s. As listed in Table 4, K_{d}s of 3±2 μM and 15±2 μM for K₁₁₅A- and K₁₁₅D-PqqT, respectively, were obtained from these studies.

Owing to the decreased affinities of K₁₁₅A- and K₁₁₅D-PqqT for PQQ (Table 4), samples used to assess metal binding were prepared with sub-stoichiometric amounts of PQQ such that >98% of PQQ are bound to PqqT during the experiments. Additional care was taken to ensure that metal-PQQ interactions observed spectroscopically occur within the PqqT fold rather than in solution: evidence for precipitation of insoluble PQQ-La³⁺ adducts were not observed spectroscopically or by inspection, and application of samples to 3 kDa MWCO centrifugal filters followed by dilution to their original volumes, revealed no optical changes by UV-vis absorbance spectroscopy. With these experimental requirements met, a series of titrations were performed, by adding Ca²⁺ or La³⁺ to PQQ⊂K₁₁₅A-PqqT, and PQQ⊂K₁₁₅D-PqqT (FIG. 2) and monitoring by UV-vis absorption and emission spectroscopies.

TABLE 4
Binding Thermodynamics Determined by ITC for Addition
of PQQ into WT-, K₁₁₅A-, and K₁₁₅D-PqqT.^{a}

| Variant | K_{d} (μM) | ΔH° (kJ/mol) | ΔS° (J · mol⁻¹ · K⁻¹) | n |
|---|---|---|---|---|
| WT-PqqT | 6 ± 4 × 10⁻² | 34 ± 2 | 250 ± 4 | 1.2 ± 0.1 |
| K₁₁₅A-PqqT | 3 ± 2 | 9.9 ± 0.7 | 140 ± 1 | 0.9 ± 0.1 |
| K₁₁₅D-PqqT | 15 ± 2 | 15.4 ± 0.7 | 145 ± 2 | 1.22 ± 0.08 |

^{a}Values reported were determined as described in the ITC Analysis section above.

As discussed above and shown in FIGS. 2A and 2E, UV-vis absorbance changes observed upon addition of metal ions to PQQ⊂WT-PqqT are small. With K₁₁₅A-PqqT, these changes are slightly more intense. However, as shown in FIG. 2G, the spectral changes observed upon addition of La³⁺ to PQQ⊂K₁₁₅D-PqqT are distinct. The features that emerge are consistent with previously reported absorbance spectra within ADH enzymes. Intriguingly, addition of Ca²⁺ to PQQ⊂K₁₁₅D-PqqT did not produce the analogous absorbance changes (FIG. 2C). Instead, relatively subtle changes were observed with Ca²⁺ that are more consistent with the spectral changes observed for titrations performed with PQQ⊂K₁₁₅A-PqqT. Given that the optical changes observed are for transitions associated with the PQQ cofactor, these results supported that La³⁺ closely associates with PQQ in the K₁₁₅D-PqqT variant. These results also indicated that we modified the metal binding specificity (Table 2).

Despite the success in generating an artificial PQQ-La³⁺ binding site, the titrations described above also revealed that multiple equivalents of La³⁺ bind to PQQ⊂K₁₁₅D-PqqT. The inset of FIG. 2G, which plots the absorbance changes at 352 and 383 nm upon addition of La³⁺, highlights this fact. Each of these wavelengths is an isosbestic point for a unique binding event. The first eq. of La³⁺ produces intensity changes at 383 nm that vary linearly with La³⁺ concentration (blue dots, FIG. 2G, inset). After one eq. of La³⁺ has been added, the absorbance intensity at 383 nm stops changing and continued addition of La³⁺ produces intensity changes at 352 nm instead (black dots, FIG. 2G, inset). The latter changes are consistent with the nonspecific metal binding observed with PQQ⊂WT-PqqT (FIG. 2).

Evidence that the first, tight-binding eq. of La³⁺ is the one closely associated with PQQ in K₁₁₅D-PqqT was obtained from titrations monitored by fluorescence spectroscopy. Emission from PQQ (at λₘₐₓ=488 nm) is quenched upon binding to PqqT. Upon complete quenching, subsequent addition of Ca²⁺ or La³⁺ to PQQ⊂WT-PqqT does not produce further changes. Repeating this experiment with PQQ⊂K₁₁₅A-PqqT produces a small amount of additional quenching. However, with PQQ⊂K₁₁₅D-PqqT, addition of La³⁺ produces a marked blueshift in the emission maximum to 450 nm. These results suggest that the metal ion is interacting strongly with the PQQ cofactor. Most importantly however, only the first eq. of La³⁺ produces this spectroscopic blueshift, revealing that it is only the first eq. that interacts strongly with PQQ in K₁₁₅D-PqqT. Continued addition of La³⁺ beyond 1 eq. produces subtle emission intensity changes consistent with nonspecific behavior observed in WT-PqqT.

Metal Binding Affinities of Variants Assessed by ITC. Quantitative measurements of Ca²⁺ and La³⁺ dissociation from each PqqT variant both with (Table 2), and without (Table 4) PQQ bound, were obtained by ITC. All data were handled in an identical manner by fitting to an independent binding model and including an added linear background correction to account for nonspecific interactions, as described in the Methods section and further in the SI. From these experiments, we determined that the K_{d} for La³⁺ binding in PQQ⊂K₁₁₅D-PqqT is 0.6±0.2 μM, an order of magnitude smaller than the K_{d}s measured for WT- or K₁₁₅A-variants. In contrast to this decrease in dissociation constant, the K_{d} for PQQ⊂K₁₁₅D-PqqT binding Ca²⁺ is 150±30 μM, 2-fold larger than the K_{d}s measured for WT- or K₁₁₅A-variants. Comparing these data, we observe a 250-fold higher affinity for La³⁺ over Ca²⁺ in PQQ⊂K₁₁₅D-PqqT.

One stark difference observed during the Ca²⁺ ITC experiments is that while the WT- and K₁₁₅A-PqqT variants maintain negative enthalpy changes during the titrations, the K₁₁₅D-PqqT variant showed a positive enthalpy change (FIG. 3). Ca²⁺ binding remained thermodynamically favorable in all variants, however binding in PQQ⊂K₁₁₅D-PqqT became entropically driven by nearly doubling the entropy term relative to that observed in the WT- and K₁₁₅A-PqqT variants. A similar increase in entropy was observed in La³⁺ binding experiments with K₁₁₅A- and K₁₁₅D-PqqT; both show nearly a 50 J·mol⁻¹·K⁻¹ increased in entropy change relative to WT-PqqT. One potential explanation for these observations is that upon modification of K₁₁₅, the PQQ pocket and adjoining tunnel can accommodate more water molecules that are displaced upon metal binding.

The dissociation constants of WT-, K₁₁₅A-, and K₁₁₅D-PqqT for La³⁺ in the absence of PQQ decrease from 500±350 μM, to 200±100 μM, to 25±9 μM, respectively, while binding to Ca²⁺ was not observed for any variant. Given the impact of K₁₁₅ mutations on these affinities, we hypothesize that the D₁₁₅ residue, and possibly the nearby E₈₄, D₁₁₄ and N₁₁₆ residues may participate in metal binding. In-line with this hypothesis, WT-PqqT only binds 0.2±0.2 equivalents of La³⁺ in the absence of PQQ, while K₁₁₅A-PqqT and K₁₁₅D-PqqT each bind 1.1±0.2 eq. La³⁺ (Table 3). The data collected with WT-PqqT likely report on nonspecific interactions, mixing, and dilution. As discussed above, when PQQ is bound, both K₁₁₅A-PqqT and K₁₁₅D-PqqT have stronger affinities for both Ca²⁺ and La³⁺ (Table 2). This increase in affinity for both metal ions in the presence of PQQ indicates that the each component contributes to the stabilization of the other within the binding pocket. Similar observations have been made in ADH enzymes, where metal ion coordination sites from both the protein and the PQQ cofactor contribute to binding and that both need to be present for metal binding to ADH enzymes.

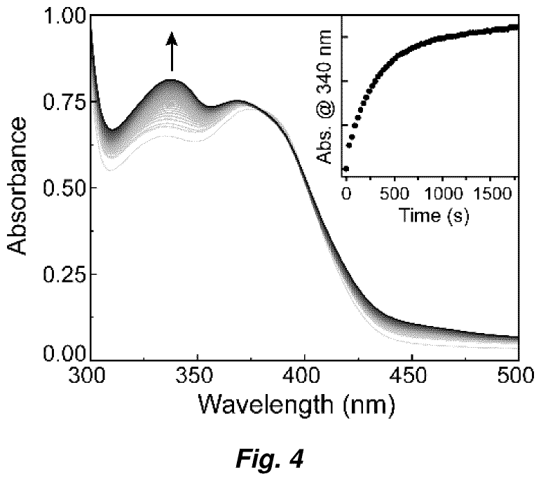

Activity Assays. To investigate whether the biomimetic ADH structural model described above was capable of biomimetic ADH function, proof-of-concept single-turnover activity assays were performed. Initial evidence for reactivity came from changes in the optical absorbance spectrum of La³⁺-PQQ⊂K₁₁₅D-PqqT in the presence of benzyl alcohol. As shown in FIG. 4 and Table 5, a new absorption feature centered at 340 nm grows in, consistent with the features of reduced PQQ observed in XoxF. No spectral changes were observed in the absence of La³⁺, PQQ, in the presence of Ca²⁺-PQQ⊂K₁₁₅D-PqqT, or with La³⁺-PQQ⊂WT-PqqT. Given the known reactivity of aldehydes with proteins, we employed a chemical trap to capture aldehyde products as their stable oxime derivatives and assessed whether the observed spectral changes correspond to ADH activity. O-((perfluorophenyl)methyl)hydroxylamine (PFBHA) was used as the chemical trap for these studies as it enabled unambiguous identification of condensation products by high resolution-mass spectrometry (HR-MS).

Performing the condensation of benzaldehyde with PFBHA provided a chemically pure standard of the expected reaction product as a mixture of E/Z-isomers, as verified by ¹H-NMR spectroscopy and HR-MS. Following the incubation of Mⁿ⁺-PQQ⊂PqqT (Mⁿ⁺=La³⁺ or Ca²⁺) with excess benzyl alcohol, equivolume methanolic solutions of PFBHA were added to the reaction mixtures to increase the miscibility of benzaldehyde, and drive protein precipitation. Centrifugation to remove solid protein products was followed by injection of the reaction supernatant onto a liquid chromatography (LC) column and the eluted contents were detected by UV absorption spectroscopy and MS. Only in the presence of La³⁺-PQQ⊂K₁₁₅D-PqqT (and not in the absence of La³⁺ or PQQ, in the presence of Ca²⁺-PQQ⊂K₁₁₅D-PqqT or La³⁺-PQQ⊂WT-PqqT) was the expected product observed. The elution times and MS analyses of reaction products were identical to those of the chemically prepared standard, albeit with different E/Z isomeric ratios. These proof-of-concept single-turnover studies support ADH activity in the ArM developed herein.

TABLE 5
Summary of assays analyzed by LC-MS.

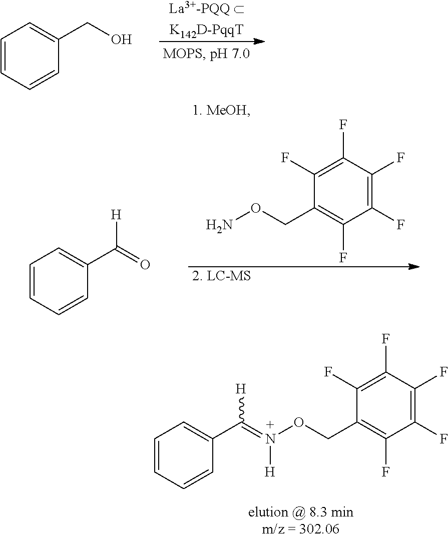

| | | | | elution | m/z |
|---|---|---|---|---|---|
| PqqT variant | PQQ | Ca³⁺ | La³⁺ | 8.3 min | 302.06 |
| WT | √ | x | √ | x | x |
| K₁₁₅D | √ | √ | x | x | x |
| K₁₁₅D | x | x | √ | x | x |
| K₁₁₅D | √ | x | √ | √ | √ |

### Discussion

First reported in 2019 (Biochemistry 2019, 58, 2665), the periplasmic PQQ-binding protein examined here is upregulated in M. extorquens AM1 in the presence of La³⁺ (3). Obtaining the crystal structure of PQQ⊂WT-PqqT (FIG. 1a) revealed that K₁₁₅ forms three H-bond interactions with PQQ, precisely analogous to coordinate bonds between La³⁺ and Ca²⁺ and PQQ in XoxF and MxaF, respectively. K₁₁₅ clearly plays a substantial role in binding PQQ, as variation at that position to Ala or Asp results in 50- and 150-fold decrease in affinity of PqqT for PQQ, respectively. Not only is this residue important for binding PQQ, it also likely plays a critical role in shielding the tridentate binding site of PQQ (O of C5, N6, and the CO₂⁻ of C7) from advantageously coordinating metals as the protein traverses the periplasm. These findings suggest that stabilization of the PQQ cofactor requires a cationic interaction at this position. Through relatively minor changes to this PQQ binding site, we were able to convert the Brønsted acidic cation site into a Lewis acidic cation-binding site. The resultant ArM mimics key features of the active sites of PQQ-dependent ADH enzymes, including reproduction of the spectroscopic signatures associated with their active sites, and catalytic activity.

Though the affinity of K₁₁₅D-PqqT for PQQ was lowered by 250-fold relative to the WT affinity (from K_{d}=60 nM to 15 μM), the new site binds La³⁺ with a K_{d}=0.6±0.2 μM and Ca²⁺, with a K_{d}=150±30 μM in PQQ⊂K₁₁₅D-PqqT, demonstrating a selectivity for La³⁺ over Ca²⁺. This specificity was surprising given the subtle differences in effective ionic radii of the two metal ions: 1.16 vs 1.12 Å, respectively. However, the result may be explained by the differences in charge density, where the introduction of the K₁₁₅D mutation adjacent to neighboring N₁₁₆ and D₁₁₄ residues is likely creating a highly negatively charged coordination environment that is more favorable for the trivalent La³⁺ ion.

The frequency with which these two metal ions appear in structurally and functionally similar enzymes (calmodulin and lanmodulin, MxaF and XoxF), and challenges associated with separating them within industrial waste streams, drives an active interest in understanding the precise structural features giving rise to a preference for one metal ion over another. The ArM presented here is well equipped to serve in efforts toward determining, and then leveraging these factors. Bench-stable and prepared on a gram scale, PqqT is an ideal candidate for performing the relevant structure-function guided studies and given the observed selectivity toward La³⁺ over Ca²⁺, may be used to develop new bioseparation strategies.

Recent advances have produced the first example of a heterologous expression system for Ln-dependent MDH, albeit in low (2 mg/L) yield. More typically, these enzymes have been expressed in their native organisms, where careful consideration of gene construction, regulation of metal content, and the presence of PQQ biosynthetic machinery are required for production in the laboratory. Accordingly, the ArM construct presented here is primed to assist the mechanistic interrogation of ADH catalysis. Though it can never be said with full certainty that a model proves aspects of a natural system, the structure-function correlations derived from ArM models have repeatedly provided key insights to these ends.

Using a combination of mutagenesis, coordination chemistry, structural, biophysical, and spectroscopic methods, we are now able to investigate key questions regarding ADH catalysis, mechanism, and metal ion dependencies. With the production of formaldehyde from methanol exceeding 20 million tons annually via reactors operating at 300-700° C., the development of a biocatalytic system that operates in water at room temperature has the potential to massively impact our energy expenditures in this area.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

General Considerations. Unless specified otherwise, all samples were prepared using distilled deionized water (ddH₂O) (18 MΩcm⁻¹) for all aqueous solutions and other uses of water. Inorganic salts and protein samples were prepared in buffer containing 30 mM HEPES, 100 mM NaCl, pH 7.0 referred to as the assay buffer.

Materials. Pyrroloquinoline quinone (PQQ), La(NO₃)₃·6H₂O, CeCl₃, NdCl₃, and GdCl₃, were obtained from Oakwood Chemicals; LaCl₃ and O-((perfluorophenyl)methyl)hydroxylamine (PFBHA) hydrochloride was purchased from Sigma Aldrich. Ni-NTA resin was obtained from Cube BioTech and Thermo Scientific. HEPES buffer, DNAse, Pierce protease inhibitor tablets (catalog number: A32963), tryptone, yeast extract, glycerol, potassium phosphate and citric acid were purchased from Fisher Scientific. MOPS, Kanamycin monosulfate and isopropylthio-β-d-galactoside (IPTG) were purchased from GoldBio. DNA oligos and gene fragments were ordered through Integrated DNA Technologies. Gibson Assembly materials and DH5α were acquired from New England Biolabs. BL21(DE3) ultracompetent cells were obtained from Promega. TEV Protease plasmid was obtained from the van der Donk lab at UIUC. All chemicals were used as ordered without further purification unless stated otherwise. PQQ and PqqT concentrations were calculated from the following extinction coefficients: ε₃₂₂(PQQ)=8,963 M⁻¹ cm⁻¹(1), and ε₂₈₀(PqqT)=32,430 M⁻¹ cm⁻¹(1). Purity of PqqT was determined by SDS-PAGE analysis, and PQQ binding activity was determined by the spectrophotometric assay described briefly below. All experiments were performed in assay buffer containing: 30 mM HEPES, 100 mM NaCl, pH 7.0 with the exception of the activity assay buffer which contained 30 mM MOPS, 100 mM NaCl, pH 7.0.

Protein expression and purification. All PqqT variants were obtained in high quantity (˜150 mg/L) and purity by using a His₆-affinity tag and cytosolic expression (see primers used in Table 6). Overexpression was performed using BL21(DE3) E. coli cell line grown in TB media. Following expression and purification by standard methods, the His₆-tag was cleaved using TEV protease, and the resultant constructs were confirmed by mass spectrometry. All subsequent experiments were performed in assay buffer containing: 30 mM HEPES, 100 mM NaCl, pH 7.0.

TABLE 6
Primers Used for Linearization and Mutational Studies of PqqT.

| Name | Sequence (sites of sequence changes shown in bold) | SEQ ID NO |
|---|---|---|
| Linearize-FWD | 5′-CATCATCACCATCATCACTAA-3′ | SEQ ID NO: 2 |
| Linearize-REV | 5′-ACCATTGCCAGACGC-3′ | SEQ ID NO: 3 |
| K₁₁₅A-FWD | 5′-GCCCTTGACGCGAACTGGATTC-3′ | SEQ ID NO: 4 |
| K₁₁₅A-REV | 5′-CCCTCCGGCGACGCCAAG-3′ | SEQ ID NO: 5 |
| K₁₁₅D-FWD | 5′-GCCCTTGACGACAACTGGATTCTGC-3′ | SEQ ID NO: 6 |
| K₁₁₅D-REV | 5′-CCCTCCGGCGACGCCAAG-3′ | SEQ ID NO: 7 |

Protein Crystallization and Structure Solution. Protein crystals were prepared by sitting drop vapor diffusion with a crystallization solution of 0.1 M citric acid, 3.2 M NaCl, pH 3.8. An initial structure, which was solved using molecular replacement with a manicured AlphaFold model, was used as a molecular replacement model for the presented PQQ-bound PqqT and PQQ/Gd³⁺-bound PqqT structures. Both structures depict only the biological monomer in the asymmetric unit. Gd³⁺ sites were assigned by analysis of anomalous maps collected at 1.3776 Å, and represent the lowest R_{free} and R_{work} values compared to when those sites were assigned as other ions (or water molecules) present in the experiment.

K_{d} Measurements. Samples were prepared such that >98% of PQQ was bound to PqqT. In all experiments, 300 μL samples of PqqT/PQQ were inserted into the ITC sample cell and 150 μL of metal solution was added to the titrant syringe, where both of these solutions were prepared in the matched assay buffer. All data were collected at ambient temperature and pressure. Model fitting was performed by combining an independent binding model with a blank (linear or constant) model to evaluate PQQ or metal binding, and residual heats to determine K_{d} and stoichiometry of titrant-protein relationships.

$$\frac{dQ}{{V}_{0} {dL}_{tot}} = Δ H° ( \frac{1}{2} + \frac{1 - \frac{1 + r}{2} - \frac{{L}_{r}}{2}}{{( {L}_{r}^{2} - 2 {L}_{r} ( 1 - r ) + {( 1 + r )}^{2} )}^{\frac{1}{2}}} )$$

$$where :$$

$${V}_{0} = initial volume$$

$$r = \frac{1}{{KP}_{tot}}$$

$$K = \frac{[ P L ]}{[ P ] [ L ]}$$

$$P = free protein$$

$$L = free ligand$$

$$PL = ligand bound to protein$$

$${L}_{tot} = total ligand$$

$${P}_{tot} = total protein$$

$$\frac{dQ}{{dL}_{tot}} = change in heat with respect to change in total ligand$$

$$Δ H ° = change in enthalpy$$

$${L}_{r} = \frac{{L}_{tot}}{{P}_{tot}}$$

Optical Spectroscopy. Metal ion titrations performed by fluorescence spectroscopy used λₑₓ=375 nm and λ_{det}=488 nm. Stoichiometries were assessed by normalizing spectra to the λₘₐₓ=488 nm value prior to addition of titrant. Samples contained 15 μM PQQ in 2.5 mL and aliquots of 1 uM PqqT or Mn+ were added in 2 uL increments. PqqT interactions with PQQ were assessed by UV-vis abs. spectroscopy monitoring 306 and 384 nm. For metal ion titrations, absorbance changes at the noted wavelengths were selected based on the unique changes observed in each case: PQQ⊂WT-PqqT (ΔA₃₆₀), PQQ⊂K115A-PqqT (ΔA₃₂₀ and ΔA₄₀₀), PQQ⊂K115D-PqqT (ΔA₃₅₂ and ΔA₃₈₃). Samples were prepared with PQQ concentrations of 100 μM and sample volumes of 3 mL.

Activity Assays. 500 μL reactions containing 100 μM PQQ, 110 μM PqqT, and 100 μM of metal ion salt in 30 mM MOPS and 100 mM NaCl at pH 7.0 were initiated by addition of BzOH to 5 mM. After incubation at room temperature (RT) for 30 min, 500 μL of MeOH containing 400 μM of PFBHA was added to the reaction mixture. Samples were vortexed for 30 s to ensure mixing, incubated at RT for 45 min, and then subjected to centrifugation for 10 min at 16,000 rcf.

Reaction supernatant were then analyzed by LC-MS using an Agilent 1260 Infinity II equipped with an AdvanceBio Peptide Plus C18 2.1×150 mm column packed with 2.7 μm resin (695775-949) and the elution gradient described in the SI. Elution products were monitored by their absorbance at 260 nm (which eluted at 8.1 and 8.3 min) and by MS counts (which elute at 8.2 and 8.4 min). HR-MS analysis performed by positive mode electrospray ionization (ESI) was used to verify product identity. In both the experimental sample and the chemically prepared control sample, both the E- and Z-isomers of the expected product were observed, albeit in different ratios.

### Example 2. Synthesis and Characterization

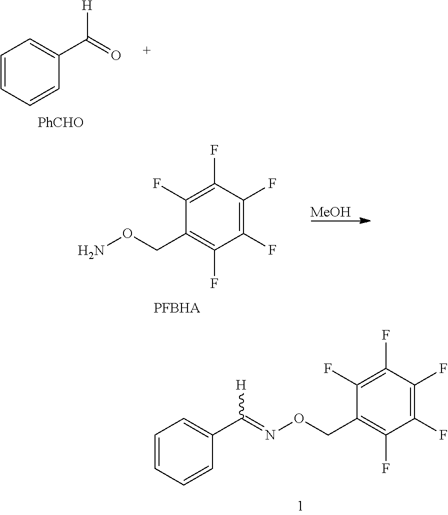 [SMILES: "PhCHO" ; "MeOH" ; O=Cc1ccccc1 ; NOCc1c(F)c(F)c(F)c(F)c1F ; Fc1c(F)c(F)c(CON=Cc2ccccc2)c(F)c1F]

Synthesis of (E)- and (Z)-benzaldehyde O-((perfluorophenyl)methyl) oxime (1). To a stirred solution of 5 mL of MeOH in a 25 mL RB flask was added 503 mg (2.00 mmol, 1.0 eq.) of O-((perfluorophenyl)methyl)hydroxylamine (PFBHA) and 266 mg (2.50 mmol, 1.2 eq) of freshly distilled PhCHO. After 45 minutes, the white precipitate (1) was filtered and dried under vacuum for 18 h. ¹H-NMR (CD₃OD, 500 MHz): 8.09 (s, 1H), 7.55 (m, 2H), 7.36 (m, 3H), 5.26 (s, 2H). ¹⁹F-NMR (CD₃OD, 600 MHz): −144.6 (dd, J₁=13 Hz, J₂=8.4 Hz, 2H), −156.9 (t, J=20 Hz, 1H), −165.5 (dd, J₁=8.4 Hz, J₂=7.0 Hz, 2H). HRMS: ESI Positive ion mode m/z [M+1] calculated: 302.0599, found: 302.0613.

Construction and Analysis of pqqT gene. The PqqT plasmid described in Table 7 was obtained as a generous gift from J. A. Cotruvo. To incorporate a TEV cleavage site before the His₆-tag, the gene was cloned into a pET-24a plasmid (Table 7). Blunt end PCR was used to linearize the plasmid (Table 6), and a TEV cleavage site was installed via Gibson Assembly (Table 8). Gene products were transformed into E. coli DH5α cells for cloning and plated on LB-agar plates containing 50 μg/mL kanamycin grown at 37° C. for 12 hours. A single colony was used to inoculate 5 mL of LB (50 μg/mL kanamycin) grown at 37° C. for 12 hours with shaking at 200 rpm. After 12 h of growth, DNA was extracted using a miniprep kit acquired from Thermo Fisher. Plasmids were submitted to ACGT for sequencing confirmation of mutation and Gibson Assembly products.

TABLE 7
Plasmids Used in this Study.

| Name | Notes |
|---|---|
| pET24a-cyto-PqqT-His | C-terminal His₆-PqqT, cytosolic expression |
| pET24a-cyto-PqqT-TEV-His | C-terminal His₆-PqqT, cytosolic expression, removal of His6-tag |

TABLE 8
Gene Block Used for Gibson Assembly.

| Name | Sequence |
|---|---|
| TEV-site | 5′-GGTGTAGGCAAATCCTTACCACCGGACCTGTATTTAGATGCGTCTG |
| gene block | GCAATGGTGAGAACCTGTACTTCCAGAGCCATCATCACCATCATCACT |
| | AAGAATTCGAGCTCCGTCGACAAGCTTGCGGC-3′ (SEQ ID NO: 8) |

TABLE 9
Wild-type sequence of the PqqT protein of Methylobacterium extorquens.

| SEQ ID NO: | Sequence (including leader sequence) |
|---|---|
| 9 | MLSRRGMLATAALSLAAMLPMARRAQAAGETFRLGVLPFGTASWEAAVIKA |
| | RGFDTANGFTLDIVKLAGNDAARIAFLGGQVDAIVGDLIFAARLGNEGRGVRF |
| | SPYSTTEGALMVPAGSPITDLKGLAGKRLGVAGGALDKNWILLRAQARETAG |
| | LELENVAQIAYGAPPLLAQKLETGELDAALLYWQFCARLEAKGFKRLISADD |
| | VMRAFGAKGAVSLIGYLYEGHTVADRGEVVRGFARASAAAKDALANEPAL |
| | WETVRPLMAAEDDATFATLKRDFLAGIPRRPIAAERADGERIYAALDRLAGA |
| | QLLGVGKSLPPDLYLDASGNG |

Cytosolic Expression and Purification of PqqT. The PqqT plasmid was transformed into E. coli BL21 (DE3) cells from Promega and plated on LB-agar plates containing 50 μg/mL kanamycin grown at 37° C. for 12 hours. A single colony was used to inoculate 50 mL of LB (50 μg/mL kanamycin in all media), grown at 37° C. for 14 hours with shaking at 200 rpm. 20 mL of the 50 mL culture was used to inoculate 2 L of TB medium and the culture was grown at 37° C. with shaking at 200 rpm until OD₆₀₀=0.5. Overexpression was induced with a final concentration of 0.2 mM IPTG. Cells were cultured for another 18 h at 18° C. and then harvested by centrifugation yielding 6 g of cell paste per L culture.

PqqT was purified as previously reported, with slight variations as described below (Biochemistry 2019, 58, 2665). The cell paste was resuspended in 4 mL/g of 50 mM sodium phosphate, 10 mM imidazole, 5% glycerol, at pH 7.0 with one Pierce protease inhibitor tablet added for every 50 mL of resuspension buffer used, along with 1% Triton-X, 5 mM MgCl₂, 2 mg of DNase and 1 mg/mL lysozyme. This resuspension mixture was incubated for 2-3 hrs with stirring at 200 rpm at 4° C. Following the lysis, the suspension was centrifuged at 12,000 rpm for 45 min. The supernatant was applied to a Ni-NTA column pre-equilibrated in 50 mM sodium phosphate, 10 mM imidazole, 5% glycerol, at pH 7.0. The column was washed with 15 column volumes (CVs) of 50 mM sodium phosphate, 10 mM imidazole, 5% glycerol, pH 7.0. The protein was eluted using 5 CV of 50 mM sodium phosphate, 250 mM imidazole, 5% glycerol, pH 7.0. The protein was concentrated to 5 mL using an Amicon Ultra 15 10-kDa MWCO centrifugal filter. Subsequently, the protein was subjected to 3 rounds of 1 L dialysis in 30 mM HEPES, 100 mM NaCl, pH 7.0 to remove any adventitiously bound molecules. The purification yielded 150 mg PqqT per L culture.

To prepare protein without the poly-histidine tag, PqqT was subjected to TEV protease at a ratio of 4 mg of TEV protease for 100 mg of purified PqqT protein. The combination of TEV protease and PqqT was incubated at 4° C. for 12 h without stirring. Following the incubation with TEV protease, the mixture was centrifuged at 4,000 rpm for 5 min and the supernatant was applied to a Ni-NTA column. The column was washed with 30 mM HEPES, 100 mM NaCl, pH 7.0. PqqT eluted during the flow through and wash steps which was collected and concentrated down to a final concentration of 1 mM and 500 μL aliquots of the purified protein were flash frozen in liquid N₂ and stored at −80° C. Confirmation of TEV cleavage was performed by 15% SDS-PAGE and mass spectrometry, which support that the tag is completely cleaved, at a site between Q and S amino acids, and leaves behind amino acids ENLYFQ following the native C-terminus of the protein. With the exception of SEC-SAXS experiments, all others reported here were performed with His₆-free protein prepared in this way.

Confirmation of P [symbol] [symbol] binding after protein purification. After protein expression, PQQ-binding activity was determined by fluorometric titrations using Horiba Fluoromax Plus-C spectrometer. 1-2 μM increments of PqqT were added to 1 mL of 15 μM PQQ. Samples were excited at 375 nm and spectra were collected between 400-600 nm. Binding activity was determined by observing the quenching of λₘₐₓ=488 nm and plotting the normalized intensity change against the molar equivalents of PQQ/PqqT using OriginPro software.

### Isothermal Titration Calorimetry (ITC).

Sample preparation. For metal binding experiments it was important to ensure all PQQ in the sample cell was bound to PqqT. To achieve this condition, a sub-stoichiometric amount of PQQ was added to the PqqT sample to ensure fully bound PQQ. An initial ITC experiment was performed for each variant of PqqT to get an approximate K_{d} so that subsequent experiments could be prepared according to this strategy. Importantly, all results are reported relative to the concentration of PQQ within the sample. Samples were prepared such that >98% of PQQ was bound to PqqT. In all experiments, 300 μL samples of PqqT/PQQ were inserted into the ITC sample cell and 150 μL of metal solution was added to the titrant syringe, where both of these solutions were prepared in the matched assay buffer. All data were collected at ambient temperature and pressure. Figure captions include experimental concentrations for each variant tested.

Instrumentation. ITC measurements were performed at 25° C. on a tabletop microcalorimetry instrument from TA-Instruments, equipped with a 190 μL active cell, a 0.01 μL injection volume precision, and full titration automation.

Analysis. ITCRun and NanoAnalyze were used for data collection and analysis, respectively. Model fitting was performed within NanoAnalyze by combining an independent binding model with a blank (linear or constant) model to evaluate PQQ or metal binding, and residual heats to determine K_{d} and stoichiometry of titrant-protein relationships. This model fitting was applied to all ITC data sets. The blank models were generated by forming linear least squares fit to heats measured after the binding event to account for any heats of dilution and residual heats. The equation for an independent binding model is as follows:

$$\frac{dQ}{{V}_{0} {dL}_{tot}} = Δ H ° ( \frac{1}{2} + \frac{1 - \frac{1 + r}{2} - \frac{{L}_{r}}{2}}{{( {L}_{r}^{2} - 2 {L}_{r} ( 1 - r ) + {( 1 + r )}^{2} )}^{\frac{1}{2}}} ) \qquad ( S1 )$$

$$where :$$

$${V}_{0} = initial volume$$

$$r = \frac{1}{{KP}_{tot}}$$

$$K = \frac{[ P L ]}{[ P ] [ L ]}$$

$$P = free protein$$

$$L = free ligand$$

$$PL = ligand bound to protein$$

$${L}_{tot} = total ligand$$

$${P}_{tot} = total protein$$

$$\frac{dQ}{{dL}_{tot}} = change in heat with respect to change in total ligand$$

$$Δ H ° = change in enthalpy$$

$${L}_{r} = \frac{{L}_{tot}}{{P}_{tot}}$$

### UV-Vis Absorption Spectroscopy.

Sample Preparation. All protein concentrations were determined by molar absorptivity prior to titrations. For metal titrations, samples were prepared in a similar way to those examined by ITC, where the PqqT concentration was adjusted to ensure >98% of PQQ was bound to protein in solution.

Instrumentation. UV-vis absorption spectra were obtained on an Agilent Technologies Spectrophotometer (Cary 8454) at 25° C.

Analysis. For determination of PqqT interactions with PQQ, the wavelengths of 306 nm and 384 were monitored for changes in absorbance. During metal titrations, in PQQ⊂WT-PqqT experiments, absorbance changes at 360 nm were monitored, for the PQQ⊂K₁₁₅A-PqqT experiments, absorbance changes at 320 nm and 400 nm were monitored, and for PQQ⊂K₁₁₅D-PqqT experiments, absorbance changes at 352 nm and 383 nm were monitored. These wavelengths were selected because they are isosbestic points in each case. All volume changes accrued during titrations were accounted for within the analysis. All data analysis and plotting was done in OriginPro software.

### Fluorescence Spectroscopy Experimental Protocols.

Sample Preparation: For titrations in which metal was added to PQQ⊂X-PqqT, samples were prepared in a similar way as those used for ITC, where the PqqT variant concentration was adjusted such that >98% of PQQ was bound during the titration according to the calculated K_{d}. Specific experimental conditions are explained within figure captions that report fluorescence titration data.

Instrumentation: Emission spectra were obtained using Horiba Fluoromax Plus-C spectrometer, at 25° C.

Analysis. For determination of metal interaction, samples were excited at λₑₓ=375 nm while observing changes in λₘₐₓ=488 nm. To observe stoichiometric interactions, spectra were normalized to the λₘₐₓ=488 nm value from the beginning of the titration. All data analyses and plotting was performed using OriginPro software.

### Activity Assay.

Sample Preparation: 500 μL reactions containing 100 μM PQQ, 110 μM PqqT, and 100 μM of metal ion salt in 30 mM MOPS and 100 mM NaCl at pH 7.0 were initiated by addition of BzOH to 5 mM. After incubation at room temperature (RT) for 30 min, 500 μL of MeOH containing 400 μM of PFBHA was added to the reaction mixture. Samples were vortexed for 30 s, allowed to incubate for 45 min at RT, then subjected to centrifugation for 10 min at 16,000 rcf. Reaction supernatant were then removed for analysis by LC-MS.

Instrumentation: LC-MS data were collected on an Agilent 1260 Infinity II equipped with an AdvanceBio Peptide Plus C18 2.1×150 mm, 2.7 μm particle size (695775-949) column. The column temperature was held constant at 45° C. and a gradient of: 85% water and 15% acetonitrile for 7.5 minutes, 100% acetonitrile for 36 seconds, and finally 4.9 minutes of 85% water and 15% acetonitrile with flow of 0.4 mL/min and pressure of 600 bar.

Analysis: Eluted products were monitored by UV absorbance at 260 nm and by MS counts. The expected products (E/Z-1) eluted at 8.1/8.3 min by UV-detection, and 8.2/8.4 minutes by MS detection. For both detection methods, the two eluted products were confirmed to be isomers of each other by HRMS, presumably from condensation of the aldehyde and hydroxylamine in cis- or trans-arrangements. Positive ion mode ESI-MS interrogation of the chromatographed samples confirmed product identity.

### Single Crystal X-Ray Diffraction (XRD).

Crystallization Conditions. X-ray quality single crystals were prepared by sitting drop vapor diffusion from 2 μL droplets of 40 mg/mL protein in 20 mM HEPES, 200 mM NaCl, pH 7.5 buffered solution combined with 2 μL droplet of 0.1M citric acid, 3.2M NaCl, pH 3.8 that was equilibrated against a reservoir solution of 200 μL of 0.1M citric acid, 3.2M NaCl, pH 3.8. Crystal trays were stored at 18° C. and crystals began forming after 21 days of storage. The crystals were soaked in a cryoprotectant solution of crystallization buffer containing 25% ethylene glycol for 60 s prior to freezing in liquid N₂. For the Gd³⁺ bound structure, crystals were harvested and incrementally incubated for 30 min in solutions containing 3.2M NaCl and 100 mM, 80 mM, 60 mM, 40 mM, 20 mM citric acid, successively. A final solution of 5 mM citric acid, 3M NaCl, 10 mM GdCl₃ was used for soaking Gd³⁺ prior to cryoprotecting and freezing the crystal.

Structure Solution. To solve the structure of PQQ-bound PqqT, a preliminary 1.8-Å resolution dataset was collected remotely at the Life Sciences Collaborative Access Team beamline 21-ID-D (EIGER 16M detector, Advanced Photon Source, Argonne National Laboratory). This structure was solved using molecular replacement in Phaser (part of the Phenix suite) and the above-described spliced model(7). More specifically, each lobe was used as an input component, and a search order was determined automatically. This iterative search yielded a single solution with space group P 2 2₁ 2₁ (LLG 847.423, TFZ 27.1). This solution contains one protomer, or biological monomer, per asymmetric unit and a solvent content of 32.6-percent.

This structure was not refined to completion but was subsequently used to solve the two additional reported higher resolution structures of PQQ-bound PqqT (1.46-Å resolution) and PQQ/Gd³⁺-bound PqqT (1.55-Å resolution). These datasets, unlike the former, were collected at beamline 12-2 (EIGER 16M detector) at the Stanford Synchrotron Radiation Lightsource, SLAC National Accelerator Laboratory. Both data sets were collected at 100K and at wavelengths of 0.9795 Å (PQQ-bound PqqT) and 1.3776 Å (PQQ/Gd³⁺-bound PqqT). As was performed for the initial 1.8-Å resolution dataset of PqqT, for these datasets, the diffraction images were manually curated using ADXV and then indexed, scaled, merged, and assigned a space group in the X-ray Detector Software (XDS)(12-14). Space group assignment was assisted by Pointless(15). Merged map files were imported into the Phenix suite, and as described above, phasing information was obtained using molecular replacement with the 1.8-Å resolution structure as a search model(7, 16, 17). For PQQ-bound PqqT, the molecular replacement solution had an LLG score of 7839.234 and a TFZ score of 83.9. Similarly high values of 6379.293 and 47.2 were obtained for PQQ/Gd³⁺-bound PqqT.

The resultant models of PQQ- and PQQ/Gd³⁺-bound PqqT were refined in Crystallographic Object-Oriented Toolkit (COOT) and using Phenix Refine. Prior to the first rounds of refinement, 5-percent of the observed reflections were flagged for the R_{free} test sets. Due to a difference in crystallization and soaking conditions, new R_{free} test sets were created for each high-resolution data set. The PQQ ligand was imported from the REFMAC monomer library (code PQQ) using COOT near the final stages of refinement and manually refined into the well-defined density(18). To overcome errors of the crystallographic programs confusing guanosine (REFMAC id: Gd) and Gd³⁺ (REFMAC id: GD), a single atom ligand file for Gd³⁺ was created in the Electronic Ligand Builder and Optimization Workbench (eLBOW), and imported into the structure in the late stage of refinement.

At the end of refinement, more precise modeling of the anomalous scattering was obtained by refining anomalous groups, with each atom of Gd³⁺ in its own group, and with starting values of f′=−4.120497 and f″=11.59798. Although we are aware that other components in our buffer may have weak anomalous signals at the energy of collection, (f″_{Na}=9.8973595E-02, f″_{Cl}=0.5711015) replacement of Gd³⁺ with either of these ions results in high levels of unmodeled difference density. Therefore, all three sites were assigned as Gd³⁺. After extensive modeling trials, the occupancy of each of these sites was set at 0.25. Of note, two of these sites distal to the PQQ ligand overlay with Na⁺ sites that are modeled in the PQQ-bound PqqT structure.

Completeness of refinement and quality of the final structures were analyzed using MolProbity(22). The MolProbity program was also used to evaluate geometry in the final structures. Here, rotamers are scored according to the observed prior likelihood using a rotamer library. This analysis resulted in the identification and assignment of Glu₈₄ and Lys₁₁₅ by COOT and the PDB Validation server as unlikely(18, 22). The expressed protein used for crystallization is lacking residues 1-27, and has a 6×-His tag appended to the C-terminus. The deposited structures of PQQ-bound PqqT and PQQ/Gd3+-bound PqqT, which are numbered according to the NCBI reference sequence (WP_015950683.1), are each missing the 6×-His tag, as well as residues 28, and 327-331, due to a lack of supporting density. The structure of PQQ/Gd³⁺-bound PqqT is additionally missing residue 29. All statistics for data collection and processing are summarized in Table 7.

Buried surface area analysis was calculated using the Proteins, Interfaces, Structures and Assemblies software (PDBePISA). Figures of the protein structures were made using PyMOL(23). Structural biology software used in this project were compiled and configured by SBGrid(24). The coordinates for the PQQ-bound PqqT and PQQ/Gd³⁺-bound PqqT structures have been deposited in the PDB with the accession codes 9B1U and 9B1V, respectively.

### Size Exclusion Chromatography Small Angle X-Ray Scattering (SEC-SAXS).

Sample preparation. To assess the conformational landscape of PqqT, small angle X-ray scattering (SAXS) experiments were pursued. Solution samples of 600 mM PqqT in the presence and absence of PQQ and La³⁺ were passed through a size exclusion column and then subjected directly to synchrotron radiation. These samples were prepared in WT-PqqT and such that PQQ was >98% bound when accounting for a K_{d}=60±40 nm for PQQ and K_{d}=6±1 μM for La³⁺.

Instrumentation. SEC-SAXS data were collected at the Cornell High Energy Synchrotron Source (CHESS). Samples containing 100 μL of PqqT sample were injected into a GE HPLC (ÄKTA purifier) and passed through a Superdex 200 10/300 column for buffer exchange into assay buffer and then routed directly and continuously at a rate of 0.15 mL/min into a BioSAXS flow cell for subsequent X-ray interrogation.

Analysis. SEC-SAXS data were collected in duplicate for each condition. A Guinier fit to the data provided the radius of gyration (R_{g}) and to assess the quality of the data collected. The Guinier analysis did not indicate evidence of aggregation or radiation damage. This analysis yielded R_{g}=21.14±0.05 Å in the absence of PQQ and R_{g}=20.53±0.04 Å when PQQ is bound showing a change in R_{g} of 0.61 Å. These data suggest that minimal conformational changes take place upon PQQ binding. When La³⁺ is added, there is again no change in R_{g}. A Kratky plot was then generated to assess the flexibility or degree of unfolding of the protein in solution. These data yielded a bell curve shaped plot that corresponds to a compact, globular protein, in good agreement with our XRD data. Finally, globular reconstruction of the protein was generated from the SAXs data. Overlaying the solved XRD structure shows that the SAXs data models the size and shape of the protein well. All analysis of SAX data was performed in BioXTAS RAW software and plotted in Origin graphing software. The bead model generation was created using DAMMIF through BioXTAS software.

Estimation of Hydrodynamic Radius. The PQQ-bound PqqT structure was analyzed using HydroPro, to estimate the radius of gyration from a crystal structure(29). Inputting the structure coordinates, and a molecular weight of 32.7 kDa(30) and specific volume default estimates of 0.702-1.0 cm³/g, the radius of gyration was estimated to be 19.4 Å. This value is in line with the roughly 44×58×27 Å maximum dimensions of the crystal structure, and is similar to the 20.53±0.04 Å value calculated from SEC-SAXS experiments.

Determination of PqqT Thermal Stability using Thermal Shift Assays. Thermal denaturation curves of PqqT were collected using a QuantStudio5 real-time PCR following previously described protocols. In brief, samples were prepared in triplicate to contain 10 μM enzyme, or PQQ (20 μM), PQQ (20 μM) and lanthanum (50 μM), or PQQ (20 μM) and calcium (50 μM). Additional samples were created to contain 10 μM enzyme and either 50 μM lanthanum or calcium. SYPRO orange dye, reaction buffer, and ultrapure water were added, to a final volume of 20 μL. Samples were then heated from 25 to 99° C. at a rate of 0.05° C./s. 25 to 99° C. at a rate of 0.05° C./s. For this experiment, as the temperature of the mixture is increased, the protein becomes unstable, and begins to unfold. The SYPRO orange dye, which increases in fluorescence when exposed to the previously sequestered hydrophobic patches of the protein, reports on the ongoing protein destabilization. After plotting the fluorescence curve at 570 nm, a melting temperature (Tₘ) was determined using the derivative of the fluorescence curve at 570 nm with the Applied Biosystems Protein Thermal Shift Software v1.4.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. An artificial metalloenzyme comprising:
a) a coordinated metal ion;
b) a pyrroloquinoline quinone (PQQ) redox cofactor;
c) an artificial periplasmic PQQ-binding protein (PqqT) engineered from wild type PqqT, wherein:
i) K₁₁₅ of the wild type PqqT is substituted with D (K₁₁₅D variant), A (K₁₁₅A variant), or H (K₁₁₅H variant); or
ii) Y₁₆₁ of the wild type PqqT is substituted with W (Y₁₆₁W variant); or
iii) both Y₁₆₁ and K₁₁₅ of the wild type PqqT are substituted wherein Y₁₆₁ is substituted with W and K₁₁₅ is substituted with D;
wherein PQQ is hydrogen bonded inside a cleft of the artificial PqqT.

2. The metalloenzyme of claim 1, wherein the redox cofactor is 4,5-dioxo-4,5-dihydro-1H-pyrrolo[2,3-f]quinoline-2,7,9-tricarboxylic acid.

3. The metalloenzyme of claim 1, wherein the metal ion is a lanthanide or actinide group ion, or a transition metal ion.

4. The metalloenzyme of claim 1, wherein the metal ion is a metal ion of La, Pr, Sm, Eu, Gd, Tb, Dy, Ho, Er, Tm, Yb, Lu, Ce, Nd, Y, Ca, Fe, Zn, Ti, V, Cr, Mn, Co, Ni, Cu, Mo, Ru, Rh, Pd, W, Re, Os, Ir, or Pt.

5. The metalloenzyme of claim 1, wherein the metal ion is La³⁺.

6. The metalloenzyme of claim 1, wherein the metalloenzyme is expressed in a Gram-negative bacterium; or an organism.

7. The metalloenzyme of claim 1, wherein the metalloenzyme is expressed in E. coli.

8. The metalloenzyme of claim 1, wherein the pyrroloquinoline quinone and the PQQ-binding protein are bound together in a 1:1 ratio.

9. The metalloenzyme of claim 1, wherein the metalloenzyme is the K₁₁₅D variant.

10. The metalloenzyme of claim 1, wherein the metalloenzyme comprises a biomimetic La³⁺-PQQ active site within the artificial periplasmic PQQ-binding protein.

11. A method for catalytic oxidation of an alcohol comprising:
contacting an alcohol with an artificial metalloenzyme according to claim 1;
wherein the alcohol enters an active site of the artificial metalloenzyme and is thereby catalytically oxidized to an aldehyde.

12. The method of claim 11, wherein the metalloenzyme is the K₁₁₅D variant and comprises a biomimetic La³⁺-PQQ active site within the artificial periplasmic PQQ-binding protein.

13. The method of claim 11, wherein the alcohol is methanol or benzyl alcohol.

14. The method of claim 11, wherein the alcohol is methanol and the methanol is oxidized to formaldehyde.

15. A method for selectively separating a metal ion species from waste solution comprising contacting the waste solution with an engineered organism, wherein the engineered organism comprises:
a) a pyrroloquinoline quinone (PQQ) redox cofactor;
b) an artificial periplasmic PQQ-binding protein (PqqT) engineered from wild type PqqT, wherein:
i) K₁₁₅ of the wild type PqqT is substituted with D (K₁₁₅D variant), A (K₁₁₅A variant), or H (K₁₁₅H variant); or
ii) Y₁₆₁ of the wild type PqqT is substituted with W (Y₁₆₁W variant); or
iii) both Y₁₆₁ and K₁₁₅ of the wild type PqqT are substituted wherein Y₁₆₁ is substituted with W and K₁₁₅ is substituted with D;
wherein PQQ is hydrogen bonded inside a cleft of the artificial PqqT, and when the metal ion species is present in the waste solution the engineered organism selectively binds the metal ion species and selectively separates it from other metal ions that may be present in the waste solution.

16. The method of claim 15, wherein the metal ion species when present in the waste solution is La³⁺, Pr³⁺, Pm³⁺, Eu³⁺, Gd³⁺, Tb³⁺, Dy³⁺, Ho³⁺, Er³⁺, Tm³⁺, Yb³⁺, Lu³⁺, Ce³⁺, Ce⁴⁺, Nd³⁺, Y³⁺, Ni²⁺, Co²⁺, or Cu²⁺.

17. The method of claim 15, further comprising filtering or centrifuging a selectively bound metal ion species from the waste solution.

18. A method for catalytic carbon-carbon bond formation comprising:
contacting an electrophilic alkane an aryl halide with an artificial metalloenzyme according to claim 1;
wherein the electrophilic alkane and the organic halide enter an active site of the artificial metalloenzyme and are thereby catalytically coupled together to form a product comprising a new carbon-carbon bond.

19. The method of claim 18, wherein the carbon-carbon bond is formed enantioselectively and the product comprises a new chiral center.

20. A method for catalytic enantioselective olefin hydrogenation comprising contacting an olefin and an artificial metalloenzyme according to claim 1 in an atmosphere of hydrogen gas, wherein the olefin and hydrogen gas enter an active site of the artificial metalloenzyme and the olefin is thereby enantioselectively hydrogenated.

21. An engineered organism expressing an exogenous metalloenzyme, the metalloenzyme comprising:
a) a coordinated metal ion;
b) a pyrroloquinoline quinone (PQQ) redox cofactor;
c) an artificial periplasmic PQQ-binding protein (PqqT) engineered from wild type PqqT, wherein:
i) K₁₁₅ of the wild type PqqT is substituted with D (K₁₁₅D variant), A (K₁₁₅A variant), or H (K₁₁₅H variant); or
ii) Y₁₆₁ of the wild type PqqT is substituted with W (Y₁₆₁W variant); or
iii) both Y₁₆₁ and K₁₁₅ of the wild type PqqT are substituted wherein Y₁₆₁ is substituted with W and K₁₁₅ is substituted with D;
wherein PQQ is hydrogen bonded inside a cleft of the artificial PqqT.

22. The engineered organism of claim 21, wherein the engineered organism is a bacterial cell; wherein optionally the bacterial cell is a Gram negative cell.

23. The engineered organism of claim 22, wherein the bacteria cell is E. coli.
